(19) United States
(12) Patent Application Publication
(10) Pub. No.: US 2004/0097495 A1
(43) Pub. Date: May 20, 2004

(54) Method for treating fibrotic diseases or other indications IIIC

(76) Inventors: Dilip Wagle, New York, NY (US); Martin Gall, Morristown, NJ (US); Stanley C. Bell, Narberth, PA (US); Edmond J. LaVoie, Princeton Junction, NJ (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/691,839

(22) Filed: Oct. 23, 2003

Related U.S. Application Data
(63) Continuation of application No. 10/036,857, filed on Dec. 31, 2001.
(60) Provisional application No. 60/259,294, filed on Dec. 29, 2000.
(60) Provisional application No. 60/259,238, filed on Jan. 2, 2001.
(60) Provisional application No. 60/296,246, filed on Jun. 6, 2001.

(51) Int. Cl. A61K 31/54; A61K 31/535; A61K 31/497; A61K 31/445; A61K 31/425

(52) U.S. Cl. 514/227.5; 514/383; 514/396; 514/406; 514/231.2; 514/252.1; 514/315; 514/365; 514/374; 514/242; 514/252.01; 514/255.05; 514/256; 514/336

(57) ABSTRACT

Provided is a method of treating or ameliorating certain fibrotic diseases or other indications in an animal, including a human, comprising administering an effective amount of a compound of the formula I:

Y—Ar^{⊕}.X⁻

wherein Ar^{⊕} is heteroaryl with a quaternary nitrogen, Y defines certain substitutions on the quaternary nitrogen, and X⁻ is anion.

This application claims the priority of U.S. application Ser. No. 60/296,246, filed Jun. 6, 2001, Ser. No. 60/259,238, filed Jan. 2, 2001, and Ser. No. 60/259,294, filed Dec.29, 2000.

The present invention relates to methods for treating certain fibrotic diseases or other indications, and to compounds and compositions for use in such treating.

Glucose and other sugars react with proteins by a non-enzymatic, post-translational modification process called non-enzymatic glycosylation. At least a portion of the resulting sugar-derived adducts, called advanced glycosylation end products (AGEs), mature to a molecular species that is very reactive, and can readily bind to amino groups on adjacent proteins, resulting in the formation of AGE cross-links between proteins. Recently a number of classes of compounds have been identified whose members inhibit the formation of the cross-links, or in some cases break the cross-links. These compounds include, for example, the thiazolium compounds described in U.S. Pat. No. 5,853,703. As AGEs, and particularly the resulting cross-links, are linked to several degradations in body function linked with diabetes or age, these compounds have been used, with success, in animal models for such indications. These indications include loss of elasticity in blood vasculature, loss of kidney function and retinopathy.

Now, as part of studies on these compounds, it has been identified that these compounds inhibit the formation of bioactive agents, such as growth factors and inflammatory mediators, that are associated with a number of indications. These agents include vascular endothelial growth factor (VEGF) and TGF[beta]. As a result, a number of new indications have been identified for treatment with agents that inhibit the formation of, or more preferably break, AGE-mediated cross-links. It is not unreasonable to infer that the effects seen are due to the removal of AGE-related molecules that provide a stimulus for the production or release of these growth factors. Removal of such molecules is believed to proceed in part due to the elimination of AGE-related cross-links that lock the AGE-modified proteins in place. Moreover, such compounds also reduce the expression of collagen in conditions associated with excess collagen production. Regardless of the mechanism, now provided are new methods of treating a number of indications.

SUMMARY OF THE INVENTION

In one embodiment, the invention relates to a method of treating or ameliorating or preventing an indication of the invention in an animal, including a human, comprising administering an effective amount of a compound of formula (I):

Y—Ar^{⊕}.X⁻ (I)

wherein Ar is a five or six membered heteroaryl ring having a first ring nitrogen and optionally second or third ring nitrogens, with the remaining ring atoms being carbon, oxygen, or sulfur, provided the first nitrogen of Ar is a quaternary nitrogen and Ar is not thiazolium, oxazolium or imidazolium. Y and other substituents on Ar are defined below.

### DETAILED DESCRIPTION OF THE INVENTION

Provided is a method of treating or ameliorating an indication of the invention in an animal, including a human, comprising administering an effective amount of (A) a compound of the formula I:

Y—Ar^{⊕}.X⁻ (I)

wherein:

a. Ar is a five or six membered heteroaryl ring having a first ring nitrogen and optionally second or third ring nitrogens, with the remaining ring atoms being carbon, oxygen, or sulfur, provided the first nitrogen of Ar is a quaternary nitrogen and Ar is not thiazolium, oxazolium or imidazolium;

b. Y is substituted on the first ring nitrogen, with the proviso that if Ar is pyrazole, indazole, (1,2,3)-triazole, benzotriazole, or (1,2,4)-triazole, the second ring nitrogen is substituted with

1. alkyl or alkoxycarbonylalkylene;

2. Ar* {wherein, consistent with the rules of aromaticity, Ar* is (and Ar², Ar³, Ar⁴ and Ar⁵ are) C₆ or C₁₀ aryl or a 5- or 6-membered heteroaryl ring, wherein 6-membered heteroaryl ring contains one to three atoms of N, and the 5-membered heteroaryl ring contains from one to three atoms of N or one atom of O or S and zero to two atoms of N, each heteroaryl ring may be fused to a benzene, pyridine (which is omitted in some embodiments), pyrimidine, pyridazine, pyrazine, or (1,2,3)triazine (wherein the ring fusion is at a carbon-carbon double bond of Ar*) (in one embodiment, Ar* is (and Ar², Ar³, Ar⁴ and Ar⁵ are) C₆ or C₁₀ aryl)}; or

3. Ar*alkyl-, Ar*C(O)alkyl-, Ar*sulfonylalkyl-, or Ar*sulfinylalkyl-; and

c. Ar can be substituted on ring carbon atoms

1. with one or more substituents independently selected from the group consisting ω-alkylenesulfonic acid, carbamoyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)— (in one embodiment, the substituents for Ar are (preferably exclusively) selected from the group consisting hydrogen, acylamino, alkanoyl, alkanoylalkyl, alkoxy, alkoxycarbonyl, alkoxycarbonylalkyl, alkyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid (—SO₃H), alkylsulfonyl (alkylSO₂—), alkylsulfinyl (alkylSO—), alkylthio, trifluoromethyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—, wherein Ar* may be substituted by one or more substituents as set forth above); or

2. two adjacent substitutions together with their ring carbons form a C₆- or C₁₀-aromatic fused ring system; or

3. two adjacent substitutions together with their ring carbons form a C₅-C₇ fused cycloalkyl ring having up to two double bonds including the fused double bond of the Ar group (in one embodiment, no double bonds except the fused double bond), which cycloalkyl ring can be substituted by one or more of the group consisting of alkyl, alkoxycarbonyl, amino, aminocarbonyl, carboxy, fluoro, or oxo (in one embodiment, multiple substituents are located on different carbon atoms of the cycloalkyl ring, except in the case of alkyl, and fluoro substituents, which can be located on the same or different carbon atoms); or

4. two adjacent substitutions together with their ring carbons form a fused five to eight membered heterocycle, wherein the ring fusion is at a carbon-carbon double bond of Ar, wherein the heterocycle consists of ring atoms selected from the group consisting of carbon, nitrogen, oxygen, and S(O)ₙ, wherein n=0,1, or 2; or

5. two adjacent substitutions together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur (in one embodiment, the substitution patterns are selected from options 1., 2. and 3.);

d. Y is:

1. a group of the formula —CH(R⁵)—R⁶ (as preferred in one embodiment)

(a) wherein R⁵ is hydrogen, alkyl-, cycloalkyl-, alkenyl-, alkynyl-, aminoalkyl-, hydroxy[C₁ to C₆]alkyl, dialkylaminoalkyl-, (N—[C₆ or C₁₀]aryl)(N-alkyl)aminoalkyl-, piperidin-1-ylalkyl-, pyrrolidin-1-ylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperazin-1-ylalkyl, piperidin-1-ylalkyl, [C₆ or C₁₀]aryl, or independently the same as R⁶ (in one embodiment, hydrogen or alkyl);

(b) wherein R⁶ is

(1) hydrogen, alkyl (which in one embodiment may be substituted by alkoxycarbonyl)-, alkenyl, alkynyl, cyano-, cyanoalkyl-, or Rs, wherein Rs is a [C₆ or C₁₀]aryl or a heterocycle containing 4-10 ring atoms of which 1-3 are heteroatoms selected from the group consisting of oxygen, nitrogen and sulfur; or

(2) a group of the formula —W—R⁷ [as preferred in one embodiment], wherein R⁷ is alkyl, alkoxy, hydroxy, or Rs [as preferred in one embodiment], wherein W is —C(═O)— or —S(O)₂—;

(3) a group of the formula —W—OR⁸ wherein R⁸ is hydrogen or alkyl,

(4) a group of the formula —CH(OH)Rs; or

(5) a group of the formula —W—N(R⁹)R¹⁰, wherein

(a) R⁹ is hydrogen and R¹⁰ is an alkyl or cycloalkyl, optionally substituted by

(i) [C₆ or C₁₀]aryl, or

(ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C1-C3)alkylenedioxy groups, or fused to a phenyl or pyridine ring, wherein the ring fusion is at a carbon-carbon double bond of the heteroaryl ring) (in one embodiment, which may or may not be in addition to the general substitutions, optionally substituted with one or more halo or (C₁-C₃)alkylenedioxy groups, or fused to a phenyl ring), or

(iii) a heterocycle containing 4-10 ring atoms of which 1-3 are heteroatoms selected from the group consisting of oxygen, nitrogen and sulfur (in one embodiment, the R¹⁰ substituents are selected from (i) and (ii)); or

(b) R⁹ is hydrogen or alkyl and R¹⁰ is Ar*; or

(c) R⁹ is hydrogen or alkyl, R¹⁰ is a heterocycle containing 4-10 ring atoms of which 1-3 are heteroatoms are selected from the group consisting of oxygen, nitrogen and sulfur; or

(d) R⁹ and R¹⁰ are both alkyl groups; or

(e) R⁹ and R¹⁰ together with N form a heterocycle containing 4-10 ring atoms which can incorporate up to one additional heteroatom selected from the group of N, O or S in the ring, wherein the heterocycle is optionally substituted with (C₆- or C₁₀)aryl, (C₆- or C₁₀)arylalkyl, or a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy (in one embodiment, ); or

(f) R⁹ and R¹⁰ are both hydrogen (in one embodiment, R⁹ and R¹⁰ are selected from the (a), (b), (e) or (f) options); or

2. —NH₂, and

e. X is a pharmaceutically acceptable anion, which may be absent if the compound provides a neutralizing salt,

(B) a pharmaceutically acceptable salt of the compound,

wherein aryl, Ar or Ar* can be substituted with, in addition to any substitutions specifically noted, one or more substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*C(O)NH—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO₃H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, thiomorpholin-4-yl, piperidin-1-yl (the “Aryl General Substituents,” where the “Ar*” recited is Ar*, Ar², Ar³, Ar⁴ or Ar⁵, as appropriate) (in one embodiment, aryl or Ar* can be substituted with, in addition to any substitutions specifically noted, one or more substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar²C(O)—, Ar²C(O)NH—, Ar²O—, Ar²-, Ar²-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid (the “Aryl Preferred General Substitutions,” where the “Ar*” recited is Ar*, Ar², Ar³, Ar⁴ or Ar⁵, as appropriate)); and

wherein heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*—, carboxy, dialkylamino, fluoro, fluoroalkyl, difluoroalkyl, hydroxy, mercapto, sulfamoyl, or trifluoromethyl (the “Heterocycle General Substituents,” where the “Ar*” recited is Ar*, Ar², Ar³, Ar⁴ or Ar⁵, as appropriate) (in one embodiment, heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylsulfonyl, alkylsulfinyl, alkylthio, Ar²C(O)—, Ar²O—, Ar²—, carboxy, fluoro, fluoroalkyl, difluoroalkyl, hydroxy, mercapto, sulfamoyl, or trifluoromethyl (the “Heterocycle Preferred General Substituents,” where the “Ar*” recited is Ar*, Ar², Ar³, Ar⁴ or Ar⁵, as appropriate)) (in one embodiment, multiple substituents are located on different atoms of the heterocyclic ring, with the proviso that alkyl, alkylcarbonyl, and fluoro substituents can be substituted on the same carbon atom of the heterocyclic ring).

In certain embodiments of the invention, if the compound of formula I has a core structure comprising a pyridinium ring having a 2-aryl-2-oxoethyl substitution at the 1 position, wherein the aryl can be substituted, and a formyl which may be substituted at the 3 position, one or both of the following applies: (1) the compound of formula VII differs from a salt of pyridinium compound having a 1-(2-aryl-2-oxoethyl), wherein the aryl can be substituted, and a formyl which may be substituted at the 3 position by at least one additional substitution at R¹⁴, R¹⁵ or R¹⁶, or (2) the aryl of 2-aryl-2-oxoethyl is phenyl and is substituted at the para position with an electron withdrawing group selected from fluoro, chloro, nitro, trifluoromethyl, and carbamoyl, and the compound used in a method of the invention is subject to the same restrictions. In certain embodiments, the compound used in a method of the invention.

The invention relates to compounds and pharmaceutical formulations including, without limitation, the compounds and formulations (compound and pharmaceutically acceptable excipient) thereof specifically recited below. In addition to the methods, compounds, and compositions thereof described herein, the invention provides methods or use in the treatments of the invention, or in the manufacture of a medicament for such therapeutic use.

Certain Fibrotic Diseases

Among the indications that can be treated with the invention are a number of indications linked to or associated with the formation of excess collagen. Among these, a number of the indications can be termed fibrotic diseases.

Such fibrotic diseases include systemic sclerosis, mixed connective tissue disease, fibrodysplasia, fibrocystic disease, sarcoidosis, myositis (e.g. polymyositis, primary idiopathic polymyositis, childhood polymyositis, dermatomyositis, childhood dermatomyositis, primary idiopathic dermatomyositis in adults, inclusion body myositis, polymyositis or dermatomyositis associated with malignant tumors). Dermatomyositis can be associated with fibrosing or hypertrophic aspects, including fibrosing alveolitis and pulmonary fibrosis. Treatment using the invention is expected to treat, prevent, reduce or ameliorate such diseases or hypertrophy, fibrotic hypertrophy or fibrosis in such diseases. Amelioration includes reducing the rate of progression of a disease. Among these fibrotic diseases are diseases that have as a manifestation fibrotic vascular intimal hypertrophy. These diseases include vasculitis (including coronary artery vasculitis), polyarteritis nodosa or temporal arteritis. Treatment using the invention is expected to treat, prevent, reduce or ameliorate vascular intimal hypertrophy in such diseases.

These fibrotic diseases further include diseases that have as a manifestation fibrotic hypertrophy of skin and/or muscle tissue. These diseases include scleroderma, eosinophilic fasciitis, discoid lesions associated with lupus or discoid lupus or surgical adhesions. Treatment using the invention is expected to treat, prevent, reduce or ameliorate such indications or hypertrophy or fibrosis of skin or muscle tissue.

Such fibrotic diseases further include diseases that have as a manifestation fibrotic hypertrophy of nerve tissue. These diseases include cerebrosclerosis, annular sclerosis, diffuse sclerosis and lobar sclerosis. Treatment using the invention is expected to treat, prevent, reduce or ameliorate such diseases, or hypertrophy, fibrotic hypertrophy or fibrosis of nerve tissue in such diseases.

These fibrotic diseases further include fibrotic lung diseases that have as a manifestation fibrotic hypertrophy or fibrosis of lung tissue. These diseases include pulmonary fibrosis (or interstitial lung disease or interstitial pulmonary fibrosis), idiopathic pulmonary fibrosis, the fibrotic element of pneumoconiosis (which is associated with exposure to environmental hazards such as smoking, asbestos, cotton lint, stone dust, mine dust and other particles), pulmonary sarcoidosis, fibrosing alveolitis, the fibrotic or hypertrophic element of cystic fibrosis, chronic obstructive pulmonary disease, adult respiratory distress syndrome and emphysema. Treatment using the invention is expected to treat, prevent, reduce or ameliorate such diseases, or hypertrophy, fibrotic hypertrophy or fibrosis in such diseases.

Such fibrotic diseases further include diseases that have as a manifestation fibrotic hypertrophy or fibrosis of prostate, liver, the pleura (e.g., pleurisy, pleural fibrosis) or pancreas. These diseases include benign prostatic hypertrophy (BPH) and fibrosis of the liver. Treatment using the invention is expected to treat, prevent, reduce or ameliorate such diseases, or hypertrophy, fibrotic hypertrophy or fibrosis in such diseases.

These fibrotic diseases further include diseases that have as a manifestation fibrotic hypertrophy or fibrosis of the bowel wall, such as inflammatory bowel disease, including Crohn's disease. Treatment using the invention is expected to treat, prevent, reduce or ameliorate such diseases, or hypertrophy, fibrotic hypertrophy or fibrosis in such diseases.

Arteriosclerosis, Atherosclerosis, Stiff Vessel Disease, Peripheral Vascular Disease, Coronary Heart Disease, Stroke, Myocardial Infarct, Cardiomvopathies, Restenosis

Arteriosclerosis is a disease marked by thickening, hardening, and loss of elasticity in arterial walls, of which atherosclerosis is a sub-type. Arteriosclerosis in turn falls within the genus of stiff vessel diseases. Without limitation to theory, it is believed that damage to the blood vessels of these diseases is due to AGE-caused damage, either through protein cross-linking or the stimulation of bioactive agents, or both. Accordingly, the agents are used to treat, prevent, reduce or ameliorate stiff vessel disease, including arteriosclerosis and athersclerosis. Peripheral vascular disease is an indication that overlaps with atherosclerosis but also covers disease which is believed to have a stronger inflammatory component. First agents are used to treat, prevent, reduce or ameliorate peripheral vascular disease. Coronary heart disease is a form of atherosclerosis of the coronary arteries. First agents are used to treat, prevent, reduce or ameliorate coronary heart disease.

When the heart pumps blood into the vascular system, the ability of the arteries to expand helps to push blood through the body. When arteries become stiff, as they do in the natural process of aging, the ability of the arteries to expand is diminished and also has consequences for the heart. The heart has to work harder to pump the blood into the stiff arteries, and eventually hypertrophies (enlarges in size) to accomplish this. A hypertrophied heart is an inefficient pump, and is one of the disorders that leads to congestive heart failure. One compound believed to work by a mechanism shared by the compounds of the invention, 3-[2-phenyl-2-oxoethyl]-4,5-dimethyl-thiazolium salt, showed an ability to reverse the stiffness of arteries in a Phase IIa clinical trial, as measured by the ratio of stroke volume (ml) to pulse pressure (mm Hg). The potential clinical benefit of this is to lessen the effort that the heart must expend to push blood throughout the body. The effect is also believed to contribute to preventing hypertrophy and subsequent inefficiency of the heart, which inefficiency would contribute to congestive heart failure.

Stroke is a cardiovascular disease that occurs when blood vessels supplying blood (oxygen and nutrients) to the brain burst or are obstructed by a blood clot or other particle. Nerve cells in the affected area of the brain die within minutes of oxygen deprivation and loss of nerve cell function is followed by loss of corresponding bodily function. Of the four main types of stroke, two are caused by blood clots or other particles. The former two are the most common forms of stroke, accounting for about 70-80 percent of all strokes.

Blood clots usually form in arteries damaged by atherosclerosis. When plaque tears from the sheer forces of blood flowing over an uneven, rigid cap atop the plaque site, thrombotic processes become involved at the “injury” site. As a result, clots can form. First agents are used to prevent, reduce or ameliorate the risk of stroke in patients who have suffered previous strokes or have otherwise been identified as at risk.

First agents can also be used to treat, prevent, reduce or ameliorate peripheral vascular disease and periarticular rigidity.

Treatment with the agents during the relatively immediate aftermath of a heart attack can be used to reduce the size of the myocardial infarct resulting from the heart attack. This treatment is preferably administered within six hours of the heart attack, more preferably, within three hours. While the dosages discussed below can be used with this indication, such as a dose of 0.01-4.0 mg/kg administered orally or 0.01-2.0 mg/kg administered intravenously, preferably within the time period outlined above. Preferred routes of administration include i.v. injection or i.v. drip. Thereafter, optional supplemental administrations can be made with the dosages described below.

Atherosclerosis is a disease that involves deposition of blood lipids in plaque in the arteries throughout the body. In coronary arteries, accumulation of plaque progressively leads to reduced coronary flow, with occlusion of the arteries causing focal death of cardiac tissue (myocardial infarction, heart attack). If the amount of tissue that dies is large enough, death ensures. In a Phase IIa trial, one compound believed to work by a mechanism shared by the compounds of the invention, 3-[2-phenyl-2-oxoethyl]-4,5-dimethyl-thiazolium salt, increased the amount of circulating triglycerides (lipids). Consistent with the known presence of AGEs in plaque, the result indicates that the agent had a lipid mobilizing effect on arterial plaque. Reducing local deposits of plaque should eventually lessen the risk of myocardial infarction and death due to heart attacks.

Fibrotic diseases further include diseases that have as a manifestation fibrotic hypertrophy of the heart. These diseases include endomyocardial fibrosis (wherein endocardium and subendocardium are fibrosed, such as in some manifestations of restrictive cardiomyopathy), dilated congestive cardiomyopathy (a disorder of myocardial function with heart failure in which ventricular dilation and systolic dysfunction predominate), hypertrophic cardiomyopathy (characterized by marked ventricular hypertrophy with diastolic dysfunction in the absence of an afterload demand), and other cardio-hypertrophies. In dilated congestive cardiomyopathy, typically at presentation there is chronic myocardial fibrosis with diffuse loss of myocytes. In hypertrophic cardiomyopathy, usually the interventricular septum is hypertrophied more than the left ventricular posterior wall (asymmetric septal hypertrophy). Treatment using the invention is expected to treat, prevent, reduce or ameliorate such diseases, or hypertrophy, fibrotic hypertrophy or fibrosis in such diseases.

Hypertrophies of the heart can be diagnosed and monitored by methods known in the art, such as by electrocardiogram, echocardiography or magnetic resonance imaging. Such diagnostic methods can be applied in particular for subjects having a risk factor for such hypertrophy, such as congestive heart failure, prior cardiac surgery or diabetes. In one aspect, the invention comprises identifying cardio-hypertrophy with using biophysical diagnostic tools, and administering an active agent of the invention to treat, prevent, reduce or ameliorate such diseases, or hypertrophy, fibrotic hypertrophy or fibrosis in such diseases. The invention can further include monitoring cardio-hypertrophy during the course of treatment with active agent.

Erosion or tearing of arterial wall plaque can occur due to the rough and irregular shape of the plaque as it forms from deposition of lipids and invasion of cells such as monocytes and macrophages (foam cells). When erosion occurs platelets and other components of the blood clotting system are activated, resulting in formation of a clot (thrombus). When the thrombus grows to such as state that blood flow is reduced, severe angina attacks that characterize unstable angina can occur. Plaque forms irregular shapes and in doing so creates shear stresses from the flow of blood over this irregular form. It is the irregularity of plaque shape that leads to the dislodging or tearing of the plaque, and to the subsequent invasion of reactive cells. On the surface of plaque is collagen, which is believed to contribute to the rigidity of the irregular shape. Without limitation to theory, it is believed that reducing the crosslinking of such a rigid collagen cap results in smoother blood flow, with a reduced risk of angina-causing tears. Accordingly, agents are used to treat, prevent, reduce or ameliorate unstable angina.

Faithful conduction of the electrical impulse from the sinoatrial to the atrioventricular nodes depends upon close apposition of myocardial cells. Excess production of collagen in the heart, which occurs naturally with aging but more so in diabetes and in conditions of heart disorders such as hypertension, causes an increase in the distance between myocardial cells, leading to atrial fibrillation. First agents are used to treat, prevent, reduce or ameliorate atrial fibrillation.

The fibrotic indications further include restenosis, which is the process of increasing artery closure following an operation to open the artery, such as balloon angioplasty.

Bladder Elasticity

Indications that can be treated, prevented, reduced or ameliorated with the agents include loss of bladder elasticity. Bladder elasticity is tied to the frequency of urination, and the urgency of desire to urinate. Accordingly, the invention can be used to treat, prevent, reduce or ameliorate non-obstructive uropathy, a disorder characterized by an overactive bladder that entails increased frequency of urination, a strong and sudden desire to urinate (urgency) which may also be associated with involuntary urinary leakage (urge incontinence).

Macular Degeneration

The effect of the agents in reducing levels of other endogenous bioactive agents, particularly VEGF and/or TGF[beta], is believed to underlie effectiveness against macular degeneration. Again, however, the invention is not limited to theory. Moreover, a anti-fibrotic effect or another effect against tissue hypertrophy may contribute. Treatment using the invention is expected to treat, prevent, reduce or ameliorate macular degeneration or macular edema. In one aspect of the invention, the treatment is used to treat, prevent, reduce or ameliorate the wet form of macular degeneration. In the wet form, new blood vessel growth has a greater contribution to the disease.

Amyotrophic Lateral Sclerosis (ALS)

ALS is associated with degradations of the motor neuron system and/or the posterior column of the spinal cord. In ALS patients, these structures tend to stain with AGE-reactive antibodies. Treatment using the invention is expected to treat, prevent, reduce or ameliorate ALS.

Rheumatoid Arthritis, Osteoarthritis, Bone Resorption

It is believed, without limitation to such theory, that reducing AGE accumulation at the joints affected by rheumatoid arthritis or osteoarthritis reduces stimulation of the production of cytokines involved in inflammatory processes of the disease. Treatment using the invention is expected to treat, prevent, reduce or ameliorate rheumatoid arthritis or osteoarthritis. Similarly, it is believed that reducing AGE accumulation at bone reduces stimulation of bone resorption. Accordingly, the invention is used to treat, prevent, reduce or ameliorate osteorporosis, bone loss or brittle bone.

Dialysis

The agents can be administered as part of a dialysis exchange fluid, thereby preventing, limiting or ameliorating the damage to tissue caused by the sugars found in such exchange fluid. For example, agents are expected to prevent, limit or ameliorate the stiffening and sclerosing of peritoneal tissue that occurs in peritoneal dialysis, as well as prevent, limit or ameliorate the formation of new blood vessels in the peritoneal membrane. In hemodialysis, agents are expected to prevent, limit or ameliorate the stiffening and sclerosing of red blood cells and vasculature resulting from exposure to the sugars exchanged into the blood during dialysis. Exchange fluids for peritoneal dialysis typically contain 10-45 g/L of reducing sugar, typically 25 g/L, which causes the formation of AGEs and consequent stiffening and degradation of peritoneal tissue. Similarly, hemodialysis fluids typically contain up to about 2.7 g/L of reducing sugar, typically 1 to 1.8 g/L. Thus, the invention provides methods by which the agents are provided in these fluids and thereby prevent, limit or ameliorate the damage that would otherwise result. Alternatively, the invention provides methods whereby the agents are administered by the methods described below to prevent, limit or ameliorate such damage from dialysis. In hemodialysis, the exchange fluid preferably contains 0.006-2.3 mg/L of an agent of the invention, more preferably, 0.06 to 1.0 mg/L. In peritoneal dialysis, the exchange fluid preferably contains 0.01 to 24 mg/L of an agent of the invention, or preferably, 1.0 to 10 mg/L.

In one embodiment, preventing or ameliorating is effected with a second agent. A preferred route of administration is inclusion in the dialysis fluids. In hemodialysis, the exchange fluid preferably contains 0.125 to 2.5 mg/L of aminoguanidine, more preferably, 0.2 to 1.0 mg/L. In peritoneal dialysis, the exchange fluid preferably contains 1.25 to 25 mg/L of aminoguanidine, or preferably, 2.0 to 10 mg/L. In a preferred aspect of the invention, the agents are initially administered, and subsequently second agents are used to moderate or limit damage thereafter.

Asthma

It is believed, without limitation to such theory, that the agents or second agents act to prevent, reduce or ameliorate the small but significant thickening of the lung airways associated with asthma. Moreover, the agents are believed to reduce stimulation of the production of cytokines involved in inflammatory processes of the disease. Accordingly, the agents are used to treat, prevent, reduce or ameliorate asthma. In this embodiment, one preferred route of administration is pulmonary, such as via an aerosol, though peroral administration is also preferred.

Carpal Tunnel Syndrome

It is believed, without limitation to such theory, that the agents act to prevent, reduce or ameliorate fibrotic and cytokine-induced elements of carpal tunnel syndrome. Accordingly, the agents are used to treat, prevent, reduce or ameliorate carpal tunnel syndrome.

Fibrotic diseases also include Dupuytren's contracture, a contracture of the palmar fascia often causing the ring and little fingers to bend into the palm. Treatment using the invention is expected to treat, prevent, reduce or ameliorate Dupuytren's contracture, or hypertrophy, fibrotic hypertrophy or fibrosis in Dupuytren's contracture.

In these embodiments, one preferred route of administration is local injection.

Periodontal Disease

The incidence of periodontal disease is higher in subjects with either insulindeficient or insulin-resistant diabetes, with consequent hyperglycemia. Again, without limitation to such theory, it is believed that the agents act to prevent, reduce or ameliorate AGE-induced cytokine action to create or exacerbate periodontal disease. Accordingly, the first or second agents are used to treat, prevent, reduce or ameliorate periodontal disease. In this embodiment, one preferred primary or supplemental route of administration is via mouthwash, or compositions adapted for delivery into the subgingival periodontal pocket (such as implants and erodible microspheres). Peroral administration is again useful. The mouthwash preferably contains 0.003-1.0 mg/L of a agent, more preferably, 0.01-0.1 mg/L.

Sickle Cell Anemia

It is believed, without limitation to such theory, that the agents act to prevent, reduce or ameliorate the restraint on blood flow caused by sickling. Again without limitation to theory, the mode of action is believed to be in reducing vascular as well as blood cell inelasticity. Accordingly, the agents are used to treat, prevent, reduce or ameliorate a sickle cell anemia.

Erectile Dysfunction

Fibrotic diseases further include diseases that have as a manifestation fibrotic disease of the penis, including Peyronie's disease (fibrosis of the cavernous sheaths leading to contracture of the investing fascia of the corpora, resulting in a deviated and painful erection). Treatment using the invention is expected to treat, prevent, reduce or ameliorate such diseases, or hypertrophy, fibrotic hypertrophy or fibrosis in such diseases.

Without limitation to theory, it is believed that the agents act to prevent, reduce or ameliorate inelasticity of tissue of the penis and/or fibrosis of tissue of the penis, such as inelasticity or fibrosis of the cavernous sheaths leading to contracture of the investing fascia of the corpora. At least partial restoration of the resulting inelasticity is believed to facilitate engorgement of the corpora cavernosa with blood. Accordingly, the agents are used to treat, prevent, reduce or ameliorate erectile dysfunction.

Limited Joint Mobility

Limited Joint Mobility (LJM) is a disorder associated with diabetes and typically involves the joints of the hands. The fourth and fifth fingers are affected initially by limitation of motion. AGE glycation and crosslinking of tendons (collagen) in the joints is believed to contribute to the disease. It is believed, without limitation to theory, that the agents act to prevent, reduce or ameliorate inelasticity, fibrous tissue or cytokine-induced inflammation associated with limited joint mobility. Accordingly, the agents are used to treat, prevent, reduce or ameliorate limited joint mobility.

Antineoplastic Applications

The agents inhibit the stimulated formation of bioactive agents, such as VEGF, associated with angiogenesis. Angiogenesis is critical for both normal development and the growth and metastasis of solid tumors. Accordingly, the agents are used to treat, prevent, reduce or ameliorate the growth of neoplasms by limiting the formation of blood vessels needed to sustain the neoplasms.

End Stage Renal Disease, Diabetic Nephropathy

Diabetic Nephropathy is a complication of diabetes that evolves early, typically before clinical diagnosis of diabetes is made. The earliest clinical evidence of nephropathy is the appearance of low but abnormal levels (>30 mg/day or 20 μg/min) of albumin in the urine (microalbuminuria), followed by albuminuria (>300 mg/24 h or 200 μg/min) that develops over a period of 10-15 years. In patients with type 1 diabetes, diabetic hypertension typically becomes manifest early on, by the time that patients develop microalbuminuria. Once overt nephropathy occurs, the glomerular filtration rate (GFR) falls over several years resulting in End Stage Renal Disease (ESRD) in 50% of type 1 diabetic individuals within 10 years and in >75% of type 1 diabetics by 20 years of onset of overt nephropathy. Albuminuria (i.e., proteinuria) is a marker of greatly increased cardiovascular morbidity and mortality for patients with either type 1 or type 2 diabetes.

Without limitation to theory, it is believed that damage to the glomerular and blood vessels of the kidney is due to AGE-caused damage, either through protein cross-linking or the stimulation of bioactive agents, or both. Accordingly, the agents are used to treat, prevent, reduce or ameliorate damage to kidney in patients at risk for ESRD. The agents can also be used to treat, prevent, reduce or ameliorate glomerulosclerosis.

Hypertension, Isolated Systolic Hypertension

Cardiovascular risk correlates more closely with the systolic and the pulse pressure than with the diastolic pressure. In diabetic patients, the cardiovascular risk profile of diabetic patients is strongly correlated to duration of diabetes, glycemic control and blood pressure. Structural matrix proteins contribute to the function of vessels and the heart, and changes in the physical behavior of cardiovascular walls are believed to be important determinants of circulatory function. In elderly individuals, the loss of compliance in the aorta leads to isolated systolic hypertension, which in turn expands the arterial wall and thereby diminishes the dynamic range of elasticity. In vivo studies in rodents, canines and in primates indicate potential utility of 3-[2-phenyl-2-oxoethyl]-4,5-dimethyl-thiazolium salt in substantially ameliorating vascular stiffening. For example, in a dog model for diabetes, lower end diastolic pressure and increased end diastolic volume, indicators of ventricular elasticity, returned to a value at about the mid-point between the disease impaired value and the value for control dogs. Treatment with 3-[2-phenyl-2-oxoethyl]-4,5-dimethyl-thiazolium salt lead to a reduction in the mass of collagen in cardiovascular tissues. In situ hybridization studies demonstrate that 3-[2-phenyl-2-oxoethyl]-4,5-dimethyl-thiazolium salt reduces the expression of both Type IV collagen and TGFbeta.

Compared with that of a non-diabetic, the diabetic artery is smaller as it is stiffer. As in isolated systolic hypertension in which vessels stiffen with age and lose the dynamic range of expansion under systole. First agents are used to treat, prevent, reduce or ameliorate hypertension, including isolated systolic hypertension and diabetic hypertension. Moreover, the same benefit is anticipated for the more rare hypertensive disorder, pulmonary hypertension. Pulmonary hypertension is a rare blood vessel disorder of the lung in which the pressure in the pulmonary artery (the blood vessel that leads from the heart to the lungs) rises above normal levels and may become life threatening. The similarity in development of elevated blood pressure in the pulmonary bed with the increase in systemic blood pressure in diabetic hypertension and in isolated systolic hypertension suggests similar mechanisms are involved.

Pulse pressure is the difference between systolic and diastolic blood pressure. In a young human, systolic pressure is typically 120 mm Hg and diastolic pressure is 80 mm Hg, resulting in a pulse pressure of 40 mm Hg. With age, in many individuals pulse pressure increases, largely due to the increase in systolic pressure that results from stiff vessel disease. In individuals with pulse pressure greater than 60 mm Hg there is an increased risk of death from cardiovascular morbidities. In a Phase IIa trial, one compound believed to work by a mechanism shared by the compounds of the invention, 3-[2-phenyl-2-oxoethyl]-4,5-dimethyl-thiazolium salt, reduced pulse pressure in elderly patients with pulse pressures greater than 60 mm Hg in a statistically significant manner. This decrease in pulse pressure was believed to be due primarily to the effect of the agent on lowering the systolic blood pressure.

The agents of the invention are used to treat, prevent, reduce or ameliorate reduced vascular compliance, elevated pulse pressure, and hypertension. Moreover, the agents are used to reduce pulse pressure, increase vascular compliance, or decrease the risk of death.

Heart Failure

Congestive Heart Failure (CHF) is a clinical syndrome that entails cardiac disease of the ventricle. Diastolic dysfunction is a subset of heart failure in which the left ventricle stiffens with age. The stiffening of the left ventricle that occurs in CHF and in diastolic dysfunction is believed to result from increased crosslinking of collagen fibers with age and/or fibrosis and related hypertrophy. First agents are used to treat, prevent, reduce or ameliorate heart failure.

Retinopathy

The effect of diabetes on the eye is called diabetic retinopathy and involves changes to the circulatory system of the retina. The earliest phase of the disease is known as background diabetic retinopathy wherein the arteries in the retina become weakened and leak, forming small, dot-like hemorrhages. These leaking vessels often lead to swelling or edema in the retina and decreased vision. The next stage is proliferative diabetic retinopathy, in which circulation problems cause areas of the retina to become oxygen-deprived or ischemic. New vessels develop as the circulatory system attempts to maintain adequate oxygen levels within the retina. Unfortunately, these new vessels hemorrhage easily. In the later phases of the disease, continued abnormal vessel growth and scar tissue may cause serious problems such as retinal detachment and glaucoma. First agents are used to treat, prevent, reduce or ameliorate diabetic retinopathy. The agents can be administered by the methods described below, including by topical administration to the eye. The agents can also be administered by intravitreal implant.

Cataracts, Other Damage to Lens Proteins

AGE-mediated crosslinking and/or fibrotic processes are believed to contribute to cataract formation and formation of other damage to lens proteins. First agents are used to treat, prevent, reduce or ameliorate cataracts or other damage to lens proteins.

Alzheimer's Disease

Considerable evidence exists implicating AGEs that form in the neurofibrillary tangles (tau protein) and senile plaques (beta-amyloid peptide) in early neurotoxic processes of Alzheimer's disease. Insoluble human tau protein is likely crosslinked. Glycation of insoluble tau from AD patients and experimentally AGE-modified tau generate oxygen free radicals, resulting in the activation of transcription via nuclear factor-kappa B, and resulting in an increase in amyloid beta-protein precursor and release of amyloid beta-peptides. Thus, A.G.E.-modified tau may function as an initiator in a positive feedback loop involving oxidative stress and cytokine gene expression. First agents are used to treat, prevent, reduce or ameliorate Alzheimer's disease.

Other Indications

For reasons analogous to those set forth above, the invention is believed to be useful in treating, preventing, reducing or ameliorating diabetes or its associated adverse sequelae, and peripheral neuropathy. The agents, especially in topical form, increase elasticity and/or reduce wrinkles in skin. The agents further increase red blood cell deformability.

Combination Therapies

In cardiovascular therapies, agents can be administered concurrently or in a combined formulation with one or more antioxidants. Examples of appropriate antioxidants are vitamin A, vitamin B6, vitamin C, vitamin E, glutathione, β-carotene, α-lipoic acid, coenzyme Q10, selenium and zinc, which are administered in effective amounts as is known in the art. Thus, the invention further provides pharmaceutical compositions comprising an agent of the invention in combination with an effective amount of an antioxidant.

In treating heart failure, cardiomyopathy or heart attack, agents can be administered concurrently or in a combined formulation with one or more angiotensin converting enzyme (ACE) inhibitors, angiotensin II receptor antagonists, calcuim channel blockers, diuretics, digitalis or beta blockers. Examples of ACE inhibitors include Captopril, Enalapril, Enalaprilat, Quinapril, Lisinopril and Ramipril, which are administered in effective amounts as is known in the art. Examples of angiotensin II receptor antagonists include Losartan, Irbesartan, Eprosartan, Valsartan and Candesartan, which are administered in effective amounts as is known in the art. Examples of calcium channel blockers include Amlopdipine, Bepridil, Diltiazem, Felodipine, Isradipine, Nicardipine, Nifedipine, Nimodipine and Verapamil, which are administered in effective amounts as is known in the art. Among diuretics, preferred examples include Furosemide, Bumetanide, Torsemide, Ethacrynic acid, Azosemide, Muzolimine, Piretanide, Tripamide and Hydrochlorothiazide, which are administered in effective amounts as is known in the art. Examples of beta adrenergic antagonists include Metoprolol, Carvedilol, Bucindolol, Atenolol, Esmolol, Acebutolol, Propranolol, Nadolol, Timolol, Pindolol, Labetalol, Bopindolol, Carteolol, Penbutolol, Medroxalol, Levobunolol, Bisoprolol, Nebivolol, Celiprolol and Sotalol, which are administered in effective amounts as is known in the art. Thus, the invention further provides pharmaceutical compositions comprising an agent of the invention in combination with an effective amount of an ACE inhibitor, diuretic, digitalis, beta blocker, or combination thereof.

For treating diabetes or complications thereof, the invention further provides pharmaceutical compositions comprising an agent of the invention in combination with an effective amount of a thiazolidinedione or “glitazone” diabetes drug, such as Troglitazone, Rosiglitazone, and Pioglitazone.

In treating atherosclerosis, agents can be administered concurrently or in a combined formulation with one or more statins (HMG CoA reductase inhibitors) or cholestyramine. Examples of statins include Mevastatin, Lovastatin, Simvastatin, Pravastatin and Fluvastatin, which are administered in effective amounts as is known in the art. Thus, the invention further provides pharmaceutical compositions comprising an agent of the invention in combination with an effective amount of a statin, cholestyramine, or both.

For a number of indications discussed, including sickle cell anemia and diabetic complications, as well as wound healing and any other indication in which increased tissue perfusion is a useful means or adjunct to therapy, the agents, or aminoguanidine or other agents of the aminoguanidine class can be administered with erythropoietin, which is administered in effective amount as is known in the art. Erythropoietin includes stable forms of erythropoietin such as are marketed by Amgen (Thousand Oaks, Calif.).

For all indications, agents can be administered concurrently or in a combined formulation with aminoguanidine or other agents of the aminoguanidine class, which are administered in effective amounts as is known in the art. These agents include compounds of formula A

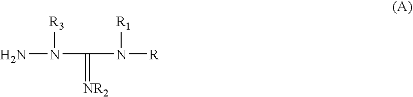

wherein R is an alkyl group, or a group of the formula —N(R⁴)(R⁵) wherein R⁴ is hydrogen, and R⁵ is an alkyl group or a hydroxyalkyl group; or R⁴ and R⁵ together with the nitrogen atom are a heterocyclic group containing 4-6 carbon atoms and, in addition to the nitrogen atom, 0-1 oxygen, nitrogen or sulfur atoms; R¹ is hydrogen or an amino group; R² is hydrogen or an amino group; R³ is hydrogen or an alkyl group, wherein R and R¹ cannot both be amino groups. Preferably at least one of R¹, R², and R³ is other than hydrogen. The compounds can be used as their pharmaceutically acceptable acid addition salts, and mixtures of such compounds. When aminoguanidine compounds are administered, they can be administered by any route of pharmaceutical administration including those discussed below for other first agents.

The method of the invention is used to treat animals, preferably mammals, preferably humans.

In accordance with the present invention, methods for administering pharmaceutical compositions containing compounds have been developed for the treating the indications of the invention. These agents are compounds of the general formula Y—Ar+X− (I), wherein Ar is a nitrogen containing, five or six-membered aromatic heterocycle as shown in the Summary section above.

Pharmaceutical compositions of the invention include administering an intraocular pressure decreasing amount of a compound of the formula I.

Compounds of the invention include compounds of the general formula Y—Ar+X−, wherein Ar is a nitrogen containing, five or six-membered aromatic heterocycle (heteroaryl). The nitrogen containing, five or six-membered aromatic heterocycle contains, consistent with the rules governing aromaticity, from 1 to 3 heteroatoms of N, O or S, with the proviso that Ar is not thiazole, oxazole, or imidazole.

The alkyl, and alkenyl groups referred to below include both C1 to C6 linear and branched alkyl and alkenyl groups, unless otherwise noted. Unless otherwise noted, alkoxy groups include linear or branched C1 to C6 alkoxy groups.

“Ar*” (and Ar², Ar³, Ar⁴, and Ar⁵) (consistent with the rules governing aromaticity) refers to a C₆ or C₁₀ aryl, or a 5 or 6 membered heteroaryl ring. The heteroaryl ring contains at least one and up to three atoms of N for the 6 membered heteroaryl ring. The 5 membered heteroaryl ring contains; (1) from one to three atoms of N, or (2) one atom of O or S and zero to two atoms of N. The aryl or heteroaryl is optionally substituted as set forth below. Nonlimiting examples of heteroaryl groups include: pyrrolyl, furanyl, thienyl, pyridyl, oxazolyl, pyrazolyl, pyrimidinyl, and pyridazinyl.

“Ar*” can be fused to either a benzene, pyridine, pyrimidine, pyridazine, or (1,2,3) triazine ring.

“Rs” refers to a C₆ or C₁₀ aryl group (wherein said aryl is optionally substituted as set forth below) or a heterocycle containing 4-10 ring members and 1-3 heteroatoms selected from the group consisting of oxygen, nitrogen and sulfur (wherein said heterocycle is optionally substituted as set forth below).

As used herein, C₆ or C₁₀ aryl groups and heterocycle containing 4 to 10 ring members are monocyclic or bicyclic.

In certain embodiments of the invention, Ar contains adjacent substitutions on ring carbons that together with their ring carbons (the carbons to which the adjacent substitution) form a fused C5 to C7 cycloalkyl ring having up to two double bonds including the fused double bond. The cycloalkyl ring can be substituted by one or more of the group consisting of alkyl, alkoxycarbonyl, amino, aminocarbonyl, carboxy, fluoro, and oxo substituents. One of ordinary skill in the art will recognized that where cycloalkyl groups contain double bonds, the sp² hybridized carbon atoms can contain only one substituent (which can not be amino- or oxo-). Sp³ hybridized carbon atoms in the cycloalkyl ring can be geminally substituted with the exception that (1) two amino groups and (2) one amino and one fluoro group can not be substituted on the same sp³ hybridized carbon atom.

In certain embodiments of the invention, Ar contains adjacent substitutions on ring carbons that together with their ring form a five to eight membered heterocycle (i.e. a bicyclic heterocycle is formed). In these embodiments the heterocycle formed by the adjacent substituents is preferably not aromatic. (Alternative embodiments refer to an aromatic heterocyclic ring, referred to as heteroaryl, formed by adjacent substitutions of Ar.) Particular compounds within embodiments containing a heterocyclic ring fused to Ar contain sulfur atoms in the fused ring. These sulfur atoms in these particular compounds can exist in various oxidation states, as S(O)ₙ, where n is 0,1, or 2.

In certain embodiments of the invention, Ar contains a Y group which can be —CH(R⁵)—R⁶. In those embodiments wherein R⁵ is alkenyl, preferably alkenyl is —C═C—R^{G}, where R^{G} is alkyl, H, or hydroxy(C₁-C₆)alkyl. In those embodiments wherein R⁵ is alkynyl, preferably alkynyl is —C═—C—R^{H}, where R^{H} is alkyl, hydrogen, or hydroxy(C₁-C₆)alkyl.

Aryl, Ar, or Ar* can be substituted with, in addition to any substitutions specifically noted, one or more substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl [alkylS(O)₂—], alkylsulfinyl [alkylS(O)—], ω-alkylenesulfonic acid [-alkylSO₃H], alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C2-C6)hydroxyalkyl, mercapto, nitro, sulfamoyl, sulfonic acid (—SO₃H), 1-pyrrolidinyl-, 4-[C6 or C10]arylpiperazin-1-yl-, 4-aryl[C6 or C10]piperidin-1-yl, azetidin-1-yl, morpholin-4-yl, thiomorpholin-4-yl, piperazin-1-yl, and piperidin-1-yl.

Heterocycles, except those of Ar and Ar* can be substituted with, in addition to any substitutions specifically noted, acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*—, carboxy, dialkylamino, fluoro, fluoroalkyl, difluoroalkyl, hydroxy, mercapto, sulfamoyl, or trifluoromethyl. Preferably, multiple substituents are located on different atoms of the heterocyclic ring , with the proviso that alkyl, alkoxycarbonyl, and fluoro substituents can be substituted on the same carbon atom of the heterocyclic ring. Heterocycles can be substituted with one or more substituents.

The halo atoms can be fluoro, chloro, bromo or iodo. Chloro and fluoro are preferred substituents for aryl substitutions.

For the purposes of this invention, the compounds of formula (I) are formed as biologically and pharmaceutically acceptable salts. Useful salt forms include the halides (particularly bromides and chlorides), tosylates, methanesulfonates, brosylates, fumarates, maleates, succinates, acetates, mesitylenesulfonates, and the like. Other related salts can be formed using similarly non-toxic, and biologically and pharmaceutically acceptable anions.

In certain embodiments of the invention, X (a pharmaceutically acceptable anion) can be absent when the molecule provides anionic moieties such as carboxylates and sulfonates. In these embodiments the compounds exist as zwitterions.

Salt formation of the nitrogen containing aromatic heterocycle (Ar) is achieved by either by alkylation or by amination (—NH₂) of a ring nitrogen atom.

Compounds of the general formula Y—Ar+. X−, can be prepared either by chemical syntheses well known in the art or by the methods described below. In addition, certain of the aromatic heterocycles, useful as intermediates for the preparation of compounds of the invention, are well-known and readily available from chemical supply houses or can be prepared by synthetic schemes specifically published therefor. The chemical reagents shown in the schemes below provide nonlimiting examples of means well known the art to carry out the reaction steps shown below.

Preferred five-membered ring heterocycles of the invention include positively charged pyrazoles, triazoles (both 1,2,3 and 1,2,4-triazoles), oxadiazoles (1,2,4), and thiadiazoles (both 1,2,3 and 1,3,4) that are alkylated at a ring N atom. Preferred compounds of the invention also include the corresponding benzo-fused analogs of the N-alkylated five-membered ring heterocycles. For example, preferred compounds of the invention includes N-alkylated indazoles, benzotriazoles and benzothiadiazoles (1,2,3).

The invention does not include positively charged analogs of the five-membered nitrogen containing heteroaromatics thiazole, oxazole, and imidazole (i.e. thiazoliums, imidazoliums, and oxazoliums).

Preferred six-membered ring heterocycles include positively charged, ring Nalkylated pyridazines, pyridines, and pyrimidines. In addition, preferred compounds of the invention include the corresponding benzo-fused analogs of the N-alkylated six-membered ring heterocycles. For example quinolines, isoquinolines, quinazolines, cinnolines, and phthalazines alkylated at a ring N atoms are preferred compounds of the invention.

In one embodiment Ar can substituted on ring carbon atoms:

1. with one or more substituents independently selected from the group consisting hydrogen, acylamino, alkanoyl, alkanoylalkyl, alkoxy, alkoxycarbonyl, alkoxycarbonylalkyl, alkyl, ω-alkylenesulfonic acid, carbamoyl, carboxy, carboxyalkyl, cycloalkyl, halo, hydroxy, (C₂C₆)hydroxyalkyl, mercapto, nitro, sulfamoyl, sulfonic acid (—SO₃H), alkylsulfonyl (alkylSO₂—), alkylsulfinyl (alkylSO—), alkylthio, trifluoromethyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—, wherein Ar* may be substituted by one or more substituents as set forth above; or

2. two adjacent substitutions together with their ring carbons form a C₆- or C₁₀-aromatic fused ring system; or

3. two adjacent substitutions together with their ring carbons form a C₅-C₇ fused cycloalkyl ring having no double bonds except the fused double bond of the Ar group, which cycloalkyl ring can be substituted by one or more of the group consisting of alkyl, amino, aminocarbonyl, carboxy, fluoro, or oxo, wherein multiple substituents are located on different carbon atoms of the cycloalkyl ring, except in the case of alkyl, and fluoro substituents, which can be located on the same or different carbon atoms [in one embodiment, the substitutions do not include amino].

In another embodiment, Y is:

1. a group of the formula —CH(R⁵)—R⁶

(a) R⁵ is hydrogen or alkyl;

(b) wherein R⁶ is

(1) hydrogen, alkyl, alkenyl, alkynyl, cyano, cyanoalkyl, or Rs; or

(2) a group of the formula —W—R⁷, wherein R⁷ is alkyl, alkoxy, hydroxy, or Rs, wherein W is —C(═O)— or —S(O)₂—;

(3) a group of the formula —W—OR⁸ wherein R⁸ is hydrogen or alkyl,

(4) a group of the formula —CH(OH)Rs; or

(5) a group of the formula —W—N(R⁹)R¹⁰, wherein

(a) R⁹ is hydrogen and R¹⁰ is an alkyl or cycloalkyl, optionally substituted by

(i) [C₆ or C₁₀]aryl, or

(ii) a 5- or 6-membered heteroaryl ring that can, in addition to the general substitutions, be optionally substituted with one or more halo or (C₁-C₃)alkylenedioxy groups, or fused to a phenyl ring, or

(b) R⁹ is hydrogen or alkyl and R¹⁰ is Ar*; or

(e) R⁹ and R¹⁰ together with N form a heterocycle wherein any heteroaryl substitution thereto can be optionally substituted, in addition to the general substitutions, with one or more halo or (C₁-C₃)alkylenedioxy; or

(f) R⁹ and R¹⁰ are both hydrogen.

In still another embodiment, Y is NH₂—.

In another embodiment, the substitutions selected from the listing above reading “wherein aryl, AR or Ar* can be substituted . . . ” do not include alkylamino, amino, dialkylamino, 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, thiomorpholin-4-yl, or piperidin-1-yl.

In still another embodiment, the substitutions selected from the listing above reading “wherein heterocycles, except those of Ar or Ar* . . . ” do not include alkylamino, amino or dialkylamino. Preferably, multiple substituents are located on different atoms of the heterocyclic ring, with the proviso that alkyl, alkylcarbonyl, and fluoro substituents can be substituted on the same carbon atom of the heterocyclic ring.

In another embodiment Y—Ar^{⊕}. X⁻ is

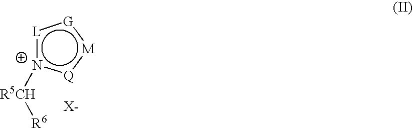

wherein G, L, M, and Q are independently O, S, N, N—R^{a}, C, C—R^{b}, C—R^{c}, C—R^{d}, wherein no more than one of G, L, M, or Q is O or S;

wherein

1. R⁵ is H;

2. R⁶is

(1) cyano or

(2) a group of the formula —W—R⁷, wherein R⁷ is alkyl or Rs, and W is —C(═O)— or —S(═O)—;

(3) a group of the formula —W—N(R⁹)R¹⁰, wherein

(a) R⁹ is hydrogen and R¹⁰ is an alkyl or cycloalkyl, optionally substituted by

(i) [C₆ or C₁₀]aryl, or

(ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, piperidin-1-yl, halo or (C₁-C₃)alkylenedioxy groups, or fused to a phenyl or pyridine ring, wherein the ring fusion is at a carbon-carbon double bond of the heteroaryl ring);

3. R^{a} is alkyl, Ar*, Ar*alkyl, alkoxycarbonylalkylene-, Ar*C(O)alkyl-, Ar*sulfonylalkyl-, or Ar*sulfinylalkyl-; and

4. R^{b}, R^{c}, and R^{d} are

(a) independently selected from the group consisting hydrogen, acylamino, acyloxyalkyl, alkanoyl, alkanoylalkyl, alkenyl, alkoxy, alkoxycarbonyl, alkoxycarbonylalkyl, alkyl, alkylamino, (C1-C3)alkylenedioxy, alkylsulfonyl, alkylsulfmyl, ω-alkylenesulfonic acid, alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO₃H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, piperidin-1-yl;

(b) wherein any two of R^{b}, R^{c}, and R^{d} are adjacent, together with their ring carbons form a C₆ or C₁₀ aromatic fused ring system;

(c) wherein any two of R^{b}, R^{c}, and R^{d} are adjacent, together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo;

(d) wherein any two of R^{b}, R^{c}, and R^{d} are adjacent, together with their ring carbons form a fused five to eight membered heterocycle, wherein the ring fusion is at a carbon-carbon double bond of Ar, wherein the fused heterocycle consists of ring atoms selected from the group consisting of carbon, nitrogen, oxygen, and S(O)ₙ wherein n=0, 1, or 2; and

(e) wherein any two of R^{b}, R^{c}, and R^{d} are adjacent, together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur.

In one embodiment of the invention defined by formula II, Ar is not tetrazole or pyrrole. In one embodiment, aryl, Ar or Ar* is substituted with, in addition to any substitutions specifically noted above, one or more substituents selected from the group consisting of hydrogen, alkyl, amino, dialkylamino, 1-pyrrolidinyl, 4-[C₆ or C₁₀]arylpiperazin-1-yl, 4[C₆ or C₁₀]arylpiperidin-1-yl, azetidin-1-yl, and morpholin-4-yl, piperidin-1-yl. The compound of formula II can be further defined in preferred embodiments as pursuant to one of the following:

| 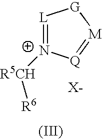 | 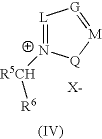 |
|---|---|
| [wherein G is O,S, or N-R^{a}; | wherein G is N or C-R^{c}; |
| M is N or C-R^{b}; | M is N or C-R^{b}; |
| Q is N or C-R^{c}; and | Q is O, S, or N-R^{a}; and |
| L is N or C-R^{d}. [It will be recognized that no | L is N or C-R^{d}. |
| more than three of G, L, M, or Q are N or N- | |
| R^{a}] | |
| 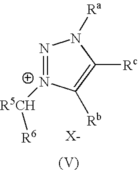 | |

[It will be recognized that no more than
two of L, G, M, Q, or R are N.]

In another embodiment, Y—Ar^{⊕}.X⁻ is

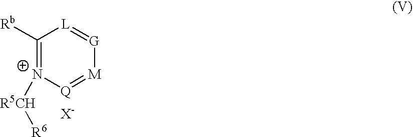

wherein L, G, M, Q, or R are independently N, C—R^{c}, C—R^{d}, C—R^{e}, C—R^{f};

wherein

1. R⁵ is H;

2. R⁶ is

(1) cyano or

(2) a group of the formula —W—R⁷, wherein R⁷ is alkyl or Rs, and W is —C(═O)— or —S(═O)—;

3. R^{b}, R^{c}, R^{d}, and R^{e} are

(a) independently selected from the group consisting hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO₃H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, piperidin-1-yl;

(b) where any two of R^{b}, R^{c}, R^{d}, and R^{e} are adjacent, together with their ring carbons form a C₆- or C₁₀-aromatic fused ring system;

(c) where any two of R^{b}, R^{c}, R^{d}, and R^{e} are adjacent, together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo;

(d) wherein any two of R^{b}, R^{c}, R^{d}, and R^{e} are adjacent, together with their ring carbons form a fused five to eight membered heterocycle, wherein the ring fusion is at a carbon-carbon double bond of Ar, wherein the fused heterocycle consists of ring atoms selected from the group consisting of carbon, nitrogen, oxygen, and S(O)ₙ wherein n=0,1, or 2;

(e) wherein any two of R^{b}, R^{c}, R^{d}, and R^{e} are adjacent, together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur, and

wherein Ar has no more than three nitrogen atoms in the ring. In one embodiment, Ar is substituted with amino, or two amino groups.

In one embodiment, any cylcloalkyl from any two adjacent substitutions to Ar that together with their ring carbons that form a C₅-C₇ fused cycloalkyl is not substituted with amino. In one embodiment, R⁵ is hydrogen, alkyl-, cycloalkyl-, alkenyl-, alkynyl-, hydroxy[C₁ to C₆]alkyl, [C₆ or C₁₀]aryl, or independently the same as R⁶. In one embodiment, any 5- or 6-membered heteroaryl ring substituted on R₁₀ is not substituted with 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl or piperidin-1-yl, and is not fused or pyridine ring. In another embodiment, any heterocycle formed from R⁹ and R¹⁰ is not substituted with 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl or piperidin-1-yl.

The invention provides a compound of formula VI:

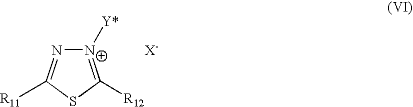

wherein

a. one of R¹¹ and R¹² is hydrogen [preferably R¹² is hydrogen] and the other is selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar², Ar²-alkyl, Ar²—O, Ar²SO²—, Ar²SO—, Ar²S—, Ar²SO₂NH—, Ar²NH, (N—Ar²)(N-alkyl)N—, Ar²C(O)—, Ar²C(O)NH—, Ar²NH—C(O)—, or (N—Ar²)(N-alkyl)N—C(O)— [in one embodiment, independently selected from hydrogen, acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, allyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid, alkylsulfonyl, alkylsulfmyl, alkylthio, trifluoromethyl, Ar², Ar²-alkyl, Ar²-O, Ar²SO₂-, Ar²SO—, Ar²S—, Ar²SO₂NH—, Ar²NH, (N—Ar²)(N-alkyl)N—, Ar²C(O)—, Ar²C(O)NH—, Ar²NH—C(O)—, and (N—Ar²)(N-alkyl)N—C(O)—];

b. Y* is a group of the formula —CH(R⁵)—R⁶ wherein (a) R⁵ is hydrogen, alkyl-, cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 1-pyrrolidinylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶ [in one embodiment, R⁵ is hydrogen or alkyl];

(b) R⁶ is

(1) cyano or Rs, wherein Rs is a [C₆ or C₁₀]aryl or a heterocycle containing 4-10 ring atoms of which 1-3 are heteroatoms selected from the group consisting of oxygen, nitrogen and sulfur;

(2) a group of the formula —W—Rs, wherein W is —C(═O)— or —S(O)ₙ— where n=1 or 2;

(3) a group of the formula —W—N(R⁹)R¹⁰, wherein

[a] R⁹ is hydrogen and R¹⁰ is an alkyl or cycloalkyl, optionally substituted by

(i) [C₆ or C₁₀]aryl, or

(ii) a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, thiomorpholin-4-yl, piperidin-1-yl, halo or (C₁-C₃)alkylenedioxy groups, or fused to a substituted phenyl or pyridine ring, wherein the ring fusion is at a carbon-carbon double bond of the heteroaryl ring [in one embodiment, such heteroaryl ring can be optionally substituted with one or more halo or (C₁-C₃)alkylenedioxy groups, or fused to a substituted phenyl], or

(iii) a heterocycle containing 4-10 ring atoms of which 1-3 are heteroatoms selected from the group consisting of oxygen, nitrogen and sulfur; or

[b] R⁹ is hydrogen or lower alkyl and R₁₀ is Ar²; or

[c] R⁹ is hydrogen or lower alkyl, and R¹⁰ is a heterocycle containing 4-10 ring atoms of which 1-3 are heteroatoms are selected from the group consisting of oxygen, nitrogen and sulfur, said heterocycle; or

[d] R⁹ and R₁₀ are both alkyl groups; or

[e] R⁹ and R¹⁰ together with N form a heterocycle containing 4-10 ring atoms which can incorporate up to one additional heteroatom selected from the group of N, O or S in the ring, wherein the heterocycle is optionally substituted with (C₆-or C₁₀)aryl, (C₆-or C₁₀)arylalkyl, or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy [in one embodiment, such heteroaryl can be optionally substituted, in addition to the general substitutions, with one or more halo or (C₁-C₃)alkylenedioxy]; or

[f] R⁹ and R₁₀ are both hydrogen; and

c. X is a pharmaceutically acceptable anion, or

(B) a pharmaceutically acceptable salt of the compound,

wherein aryl or Ar² can be substituted with, in addition to any substitutions specifically noted, one or more substituents selected from the Aryl General Substitutions [in one embodiment, one or more substituents selected from the Aryl Preferred General Substituions];

wherein heterocycles, except those of Ar², can be substituted with, in addition to any substitutions specifically noted, the Heterocyle General Substitutions [in one embodiment, the Heterocycle Preferred General Substituents];

wherein the compound of formula VI differs from a salt of 3-[2-(4-bromophenyl)-2-oxoethyl]-1,3,4-thiadiazolium by one or more of the lack or replacement of the 4-bromo substitution, or the presence of one or more additional substitutions [preferably the differences in substitutions total two or more]; and

wherein the compound of formula VI differs from a salt of 3-(phenylmethyl)-1,3,4-thiadiazolium by the presence of one or more additional substitutions [preferably the differences in substitutions total two or more].

3-[2-(4-Bromophenyl)-2-oxoethyl]-1,3,4-thiadiazolium bromide and 3-(phenylmethyl)-1,3,4-thiadiazolium chloride are described in Haug et al., Liebigs Ann. Chem. 1988(6): 605-7, as intermediates for forming spirocyclic compounds. 3-(Phenylmethyl)-1,3,4-thiadiazolium chloride is also mentioned in JP 04081597 (issued Dec. 24, 1992), it is believed as a reagent involved in converting formaldehyde to dendroketose, and in Takamizawa et al., Chem. Pharm. Bull. 18(6):1201-10, 1970, an article on the reaction of 1,3,4-thiadiazolium halides with dialkyl acylphosphonates in the presence of Et₃N to give 1,3,4-thiadiazine derivatives, accompanied by ring expansion.

The invention further provides a compound of formula VII:

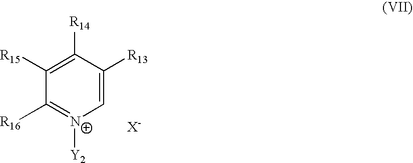

wherein

a. R¹³, R¹⁴, R¹⁵ and R¹⁶

1. are independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar³, Ar³-alkyl, Ar³—O, Ar³SO₂—, Ar³SO—, Ar³S—, Ar³SO₂NH—, Ar³NH, (N—Ar³)(N-alkyl)N—, Ar³C(O)—, Ar³C(O)NH—, Ar³NH—C(O)—, and (N—Ar³)(N-alkyl)N—C(O)—, or together R¹ and R₂ comprise methylenedioxy [in one embodiment, independently selected from hydrogen, acylamino, acyloxyalkyl, alkanoyl, alkanoylalkyl, alkenyl, alkoxy, alkoxycarbonyl, alkoxycarbonylalkyl, alkyl, (C¹-C₃)alkylenedioxy, allyl, ω-alkylenesulfonic acid, carbamoyl, carboxy, carboxyalkyl, cycloalkyl, halo, hydroxy, (C₂C₆)hydroxyalkyl, mercapto, nitro, sulfamoyl, sulfonic acid, alkylsulfonyl, alkylsulfinyl, alkylthio, trifluoromethyl, Ar³, Ar³-alkyl, Ar³—O, Ar³SO₂—, Ar³SO—, Ar³S—, Ar³SO₂NH—, Ar³NH, (N—Ar³)(N-alkyl)N—, Ar³C(O)—, Ar³C(O)NH—, Ar³NH—C(O)—, and (N—Ar³)(N-alkyl)N—C(O)-]; or

2. form, with an adjacent pair from R¹³, R¹⁴, R¹⁵ and R¹⁶, together with their ring carbons, a C₆- or C₁₀- aromatic fused ring system; or

3. form, with an adjacent pair from R¹³, R¹⁴, R¹⁵ and R¹⁶, together with their ring carbons, a C₅-C₇ fused cycloalkyl ring having up to two double bonds including the fused double bond of the pyridinium containing ring, which cycloalkyl ring can be substituted by one or more of the group consisting of alkyl, alkoxycarbonyl, amino, aminocarbonyl, carboxy, fluoro, or oxo substituents [in one embodiment, the substitutions do not include amino]; or

4. form, with an adjacent pair from R¹³, R¹⁴, R¹⁵ and R¹⁶, together with their ring carbons, a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring may be optionally substituted with one or more 1-pyrrolidinyl-, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy groups [in one embodiment, the optional substitutions are one or more halo or (C₁-C₃)alkylenedioxy groups]; or

5. form, with an adjacent pair from R¹³, R¹⁴, R¹⁵ and R¹⁶, together with their ring carbons, a five to eight membered heterocycle, wherein the heterocycle consists of ring atoms selected from the group consisting of carbon, nitrogen, and S(O)ₙ, where n=0,1, or 2;

b. Y² is a group of the formula —CH(R⁵)—R⁶ wherein

(a) R⁵ is hydrogen, alkyl-, cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 4pyrrolidin-1-ylalkyl, azetidinylalkyl, 4-alkylpiperazin-1-ylalkyl, 4alkylpiperidin-1-ylalkyl, 1-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶ [in one embodiment, R⁵ is hydrogen or alkyl];

(b) R⁶ is

(1) cyano or Rs, wherein W is —C(═O)— or —S(O)ₙ— where n=1 or 2, and Rs is a C₆ or C₁₀ aryl or a heterocycle containing 4-10 ring atoms of which 1-3 are heteroatoms selected from the group consisting of oxygen, nitrogen and sulfur;

(2) a group of the formula —W—Rs, wherein W is —C(═O)— or —S(O)ₙ— where n=1 or 2;

(3) a group of the formula —W—N(R⁹)R¹⁰, wherein

[a] R⁹ is hydrogen and R¹⁰ is an alkyl or cycloalkyl, optionally substituted by

(i) [C₆ or C₁₀]aryl, or

(ii) a 5- or 6-membered heteroaryl ring, wherein the 6-membered heteroaryl ring contains one to three atoms of N, and the 5membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, thiomorpholin-4-yl, piperidin-1-yl, halo or (C₁-C₃)alkylenedioxy groups, or fused to a phenyl or pyridine ring, wherein the ring fusion is at a carbon-carbon double bond of the heteroaryl ring [in one embodiment, the optional substitutions are one or more halo or (C₁-C₃)alkylenedioxy groups, or fused to a substituted phenyl], or

(iii) a heterocycle containing 4-10 ring atoms of which 1-3 are heteroatoms selected from the group consisting of oxygen, nitrogen and sulfur; or

[b] R⁹ is hydrogen or lower alkyl and R¹⁰ is Ar³; or

[c] R⁹ is hydrogen or lower alkyl, and R¹⁰ is a heterocycle containing 4-10 ring atoms of which 1-3 are heteroatoms are selected from the group consisting of oxygen, nitrogen and sulfur, said heterocycle; or

[d] R⁹ and R¹⁰ are both alkyl groups; or

[e] R⁹ and R¹⁰ together with N form a heterocycle containing 4-10 ring atoms which can incorporate up to one additional heteroatom selected from the group of N, O or S in the ring, wherein the heterocycle is optionally substituted with (C₆-or C₁₀)aryl, (C₆-or C₁₀)arylalkyl, or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each such heteroaryl can be optionally substituted, in addition to the general substitutions, with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy [in one embodiment, the optional substituents to the heteroaryl are one or more halo or (C₁-C₃)alkylenedioxy]; or

[f] R⁹ and R¹⁰ are both hydrogen;

c. X is a pharmaceutically acceptable anion, or

(B) a pharmaceutically acceptable salt of the compound,

wherein aryl or Ar³ can be substituted with, in addition to any substitutions specifically noted, one or more substituents selected from the Aryl General Substituents or the Aryl Preferred General Substituents;

wherein heterocycles, except those of Ar, can be substituted with, in addition to any substitutions specifically noted, the Heterocycle General Substituents or the Heterocycle Preferred General Substituents; and

wherein, if the compound of formula VII has a core structure comprising a pyridinium ring having a 2-aryl-2-oxoethyl substitution at the 1 position, wherein the aryl can be substituted, and a formyl which may be substituted at the 3 position, one or both of the following applies:

the compound of formula VII differs from a salt of pyridinium compound having a 1-(2-aryl-2-oxoethyl), wherein the aryl can be substituted, and a formyl which may be substituted at the 3 position by at least one additional substitution at R¹⁴, R¹⁵ or R¹⁶, or

the aryl of 2-aryl-2-oxoethyl is phenyl and is substituted at the para position with an electron withdrawing group selected from fluoro, chloro, nitro, trifluoromethyl, , and carbamoyl; and

wherein the compound of formula VII differs from a salt of 1-[2-(4-methylphenyl)-2-oxoethyl]-pyridinium by one or more of the lack or replacement of the methyl substitution, or the presence of one or more additional substitutions [preferably the differences in substitutions total two or more].

Sankaranarayanan, WO 01/25209 describes certain pyridinium compounds substituted on the 1 (N) position 2-aryl-2-oxoethyl substitutions and derivative of formyl at the 3 position. 1-[2-(4-methylphenyl)-2-oxoethyl]-pyridinium chloride is described in J. Med. Chem. 32: 2301-6, 1989, as an inactive member of a series of compounds that sought to explore the glucose lowering effect of, particularly, certain imidazolium compounds.

The invention further provides a compound of formula VIII:

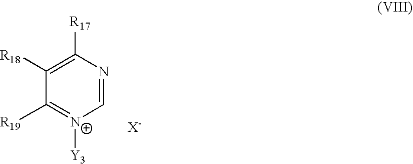

wherein

a. R¹⁷, R¹⁸ and R¹⁹

1. are independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁴, Ar⁴-alkyl, Ar⁴—O, Ar⁴SO₂—, Ar⁴SO—, Ar⁴S—, Ar⁴SO₂NH—, Ar⁴NH, (N—Ar⁴)(N-alkyl)N—, Ar⁴C(O)—, Ar⁴C(O)NH—, Ar⁴NH—C(O)—, and (N—Ar⁴)(N-alkyl)N—C(O)—, or together R₁ and R₂ comprise methylenedioxy; or

2. form, with an adjacent pair from R¹⁷, R¹⁸ and R¹⁹, together with their ring carbons, a C₆- or C₁₀- aromatic fused ring system; or

3. form, with an adjacent pair from R¹⁷, R¹⁸ and R¹⁹, together with their ring carbons, a C₅-C₇ fused cycloalkyl ring having up to two double bonds including the fused double bond of the pyridinium containing ring, which cycloalkyl ring can be substituted by one or more of the group consisting of alkyl, alkoxycarbonyl, amino, aminocarbonyl, carboxy, fluoro, or oxo substituents [in one embodiment, the substitutions do not include amino; or

4. form, with an adjacent pair from R¹⁷, R¹⁸ and R¹⁹, together with their ring carbons, a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring may be optionally substituted with one or more 1-pyrrolidinyl-, 4-[C₆ or C₁₀]arylpiperazin-1-yl, 4-[C₆ or C₁₀]arylpiperidin-₁-yl, azetidin-1-yl, morpholin-4-yl, thiomorpholin-4-yl, piperidin-1-yl, halo or (C₁-C₃)alkylenedioxy groups [in one embodiment, the optional substitutions are one or more halo or (C₁-C₃)alkylenedioxy groups]; or

5. form, with an adjacent pair from R¹⁷, R¹⁸ and R¹⁹, together with their ring carbons, a five to eight membered heterocycle, wherein the heterocycle consists of ring atoms selected from the group consisting of carbon, nitrogen, and S(O)ₙ, where n=0, 1, or 2;

b. Y³ is a group of the formula —CH(R⁵)—R⁶ wherein

(a) R⁵ is hydrogen, alkyl-, cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 1-pyrrolidin-1-ylalkyl, azetidinylalkyl, 4-alkylpiperazin-1-ylalkyl, 4alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶ [in one embodiment, R⁵ is hydrogen or alkyl];

(b) R⁶ is phenyl substituted on the para position with chloro or fluoro;

c. X is a pharmaceutically acceptable anion, or

(B) a pharmaceutically acceptable salt of the compound,

wherein aryl (including phenyl) or Ar⁴ can be substituted with, in addition to any substitutions specifically noted, one or more general substituents selected from the Aryl General Substitutions or Aryl Preferred General Substitutions; and

wherein heterocycles, except those of Ar⁴, can be substituted with, in addition to any substitutions specifically noted, the Heterocycle General Substitutions or Heterocycle Preferred General Substitutions;

wherein, in one embodiment, if Y has a core structure of phenyl substituted at the para position with chloro, then the compound of formula VIII differs from a salt of 1-[2-(4-bromophenyl)-2-oxoethyl]-5-cyano-pyrimidinium by a substitution difference of more than the cyano (which is not within the scope of R¹⁸).

1-[2-(4-chlorophenyl)-2-oxoethylide]-5-cyano-pyrimidinium, 2-(4-nitrophenyl)-2-oxoethylide)-pyrimidinium and 2-(4-nitrophenyl)-2-oxoethyl)-pyrimidinium bromide are described in U.S. Pat. Nos. 3,836,352 and 3,702,361 as herbicides. 1-[2-(4-Bromophenyl)-2-oxoethyl]-4-(4-mehtylphenyl)-pyrimidinium bromide is is available from the Sigma-Aldrich Rare Chemical Library. 1-[2-[3,4-bis(acetyloxy)phenyl]-2-oxoethyl]-4-methylthio-pyrimidinium chloride is described in EP 304155 and corresponding U.S. Pat. No. 5,013,730, as in intermediate for making cephalosporin compounds. 4-(3-Methylphenyl)-1-[2-(2-nitrophenyl)-2-oxoethyl]-pyrimidinium bromide has a registry number of 373638-66-5.

The invention further provides a compound of formula IX:

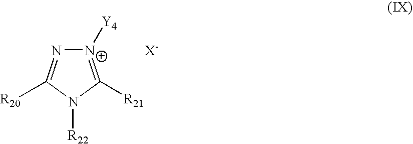

wherein

a. one of R²⁰ and R²¹ is hydrogen, and the other is selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁵, Ar⁵-alkyl, Ar⁵—O, Ar⁵SO₂—, Ar⁵SO—, Ar⁵S—, Ar⁵SO₂NH—, Ar⁵NH, (N—Ar⁵)(N-alkyl)N—, Ar⁵C(O)—, Ar⁵C(O)NH—, Ar⁵NH—C(O)—, or (N—Ar⁵)(N-alkyl)N—C(O)—;

b. R²² is acylamino, acyloxyalkyl, alkanoylalkyl, alkenyl, alkoxycarbonyl, alkoxycarbonylalkyl, alkyl, allyl, carbamoyl, carboxyalkyl, dialkylamino, (C₂C₆)hydroxyalkyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁵, Ar⁵-alkyl, Ar⁵—O, Ar⁵SO₂—, Ar⁵SO—, Ar⁵S—, Ar⁵SO₂NH—, Ar⁵NH, (N—Ar⁵)(N-alkyl)N—, Ar⁵C(O)—, Ar⁵C(O)NH—, Ar⁵NH—C(O)—, or (N—Ar⁵)(N-alkyl)N—C(O)—;

c. Y⁴ is a group of the formula —CH(R⁵)—R⁶ wherein

(a) R⁵ is hydrogen, alkyl-, cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 1-pyrrolidinylalkyl, azetidinylalkyl, 4-alkylpiperazin-1-ylalkyl, 4alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶;

(b) R⁶ is

(1) cyano;

(2) a group of the formula —W—Rs, wherein W is —C(═O)— or —S(O)ₙ— where n=1 or 2, and wherein Rs is a [C₆ or C₁₀]aryl or a heterocycle containing 4-10 ring atoms of which 1-3 are heteroatoms selected from the group consisting of oxygen, nitrogen and sulfur; and

d. X is a pharmaceutically acceptable anion, or

(B) a pharmaceutically acceptable salt of the compound,

wherein aryl (including phenyl) or Ar⁵ can be substituted with, in addition to any substitutions specifically noted, one or more general substituents selected from the Aryl General Substitutions or Aryl Preferred General Substitutions; and

wherein heterocycles, except those of Ar⁵, can be substituted with, in addition to any substitutions specifically noted, the Heterocycle General Substitutions or Heterocycle Preferred General Substitutions;

The invention further relates to the pharmaceutical compositions of the compounds specifically recited comprising such compounds with a pharmaceutically acceptable excipient, and effecting the methods of the invention with these compounds.

In general, compounds of the general formula Y—Ar+. X−, wherein Y is as described above; and Ar is a nitrogen containing five or six-membered aromatic heterocycle, can be prepared by alkylation of the heterocycle under suitable alkylating conditions. By way of example, a 3-methyl (1,2,3)thiadiazolium salts can be prepared by N-alkylation of (1,2,3)thiadiazole with suitable alkylating agents, such as methyl iodide or methyl p-toluenesulfonic acid ester (Wolff, Kopitzsch Justus Liebigs Ann. Chem., 1904, 333, 20 and Adachi, J.; Takahat, H.; Nomura, K; Masuda, K. Chem. Pharm. Bull., 1983, 31(5) 1746-1750). In another example of the invention, 1-methyl triazole can be alkylated with benzyl iodide to give a compound wherein R⁵ is hydrogen and R⁶ is phenyl and Ar is a 3-methyl-(1,2,3)triazole (i.e. 1-benzyl-3-methyltriazolium iodide) (J. Am. Chem Soc., 1955, 77, 1703).

Generally, the compounds of the invention are synthesized by reacting Ar with Y—X, where X is a leaving group.

As is known in the art, alkylation of heterocycles containing more than one nitrogen atom in the ring (e.g., pyridazine, pyrazole, thiadiazole) can often lead to isomeric mixtures of N-alkylated products. In these cases, the isomers can be separated by any separation known in the art including fractional recrystallization, column chromatography, and the like. By way of example, alkylation of 4-substituted pyridazines can lead to mixture of pyridazines as shown below in Scheme 1. The isomeric N-alkylated pyridazine can be separated by the above mentioned techniques to provide compounds of the invention.

Scheme 1

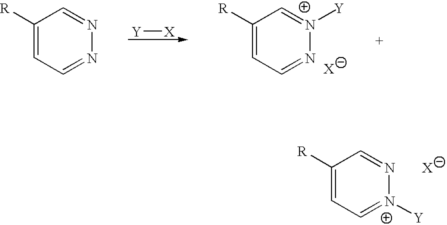

Those of ordinary skill in the art will recognize that some 5-membered aromatic heterocycles require substitution of two different nitrogen atoms in the heterocycle to effect quaternization. Five-membered ring heterocycles of this type include pyrazoles, (1,2,4)-triazoles, and (1,2,3)-triazoles (and benzofused analogs such as indazole and benzotriazole). The incorporation of one alkyl group in the heterocycle can be accomplished either by the use of a suitable N-alkylated acyclic precursor for ring formation, or by alkylation of the intact heterocycle. For instance, an alkyl pyrazole intermediate can be prepared by condensation of an alkyl hydrazine and a 1,3-dicarbonyl compound, or by alkylation with a pyrazole using suitable alkylating conditions (Scheme 2).

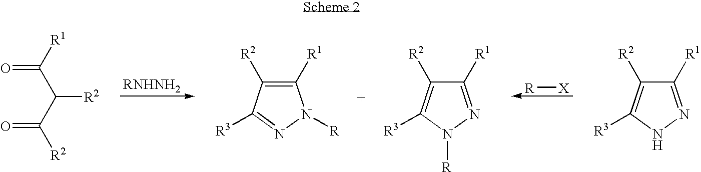

In general, compounds of the general formula Y—Ar+. X−, wherein Y is CH(R⁵)—C(O)—R⁷ (wherein R⁵ and R⁷ is as described above; G, L, M, and Q are independently C, N, S or O; and X is a halide) can be prepared according to the synthetic route depicted in Scheme 3. An acetyl derivative with a suitable α leaving group, for example, an α-halo acetyl derivative, can be used to N-alkylate a suitably substituted aromatic heterocycle. The alkylation reaction can be conducted at elevated temperatures in a suitable solvent, for example, acetonitrile, acetone, or ethanol, or without solvent.

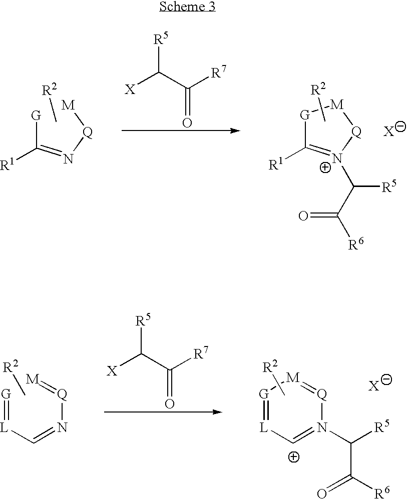

For example, a substituted 1,2,4-triazole (such as 4-phenyl substituted) can be alkylated with a substituted phenacyl bromide in acetone as shown in Scheme 4 (Surpateanu, G. G.; Vergoten, G.; Elass, A.; Surpateanu, G.; Heterocycles, 1999, 51, 2213-2220). Other 5-membered nitrogen heterocycles can be alkylated similarly.

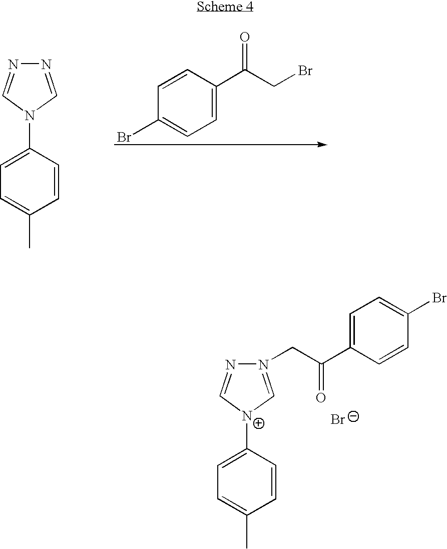

Analogously, a 1,2,4-triazole (which can be substituted) that is substituted at one ring nitrogen can be reacted with Y—X to form the charged species. For example, 1,2,4-triazole substituted at the 4 position (for example with amino, alkyl amino or alkyl) can be reacted with Y-X (for example 2-chloro-1-phenyl-ethanone).

In another example (1,3,4)-thiadiazole can be alkylated in acetonitrile with the same substituted phenacyl bromide to give 3-(4-bromophenacyl)-(1,3,4)-thiadiazolium bromide (Haug, E.; Kantlehner, W.; Hagen, H.; Speh, P.; Braeuner, H. Liebigs Ann. Chem., 1988, 605-608).

Six membered aromatic heterocycles such as pyridazine, pyrimidine, and pyridazine can be similarly alkylated with α-halo carbonyl containing reagents. For example, 1-(4-methylphenacyl)pyridazinium bromide can be prepared by reaction of equimolar amounts of 4-methylphenacyl bromide and pyridazine in refluxing acetonitrile (J. Med. Chem., 1989, 32, 2301-2306). Pyrimidines and can be prepared similarly. For example, 1-phenacylpyrimidinium bromide can be prepared by reaction of phenacyl bromide and pyrimidine (Chem. Ber., 1958, 91, 2832). Pyridine analogs, can also be prepared by this method. The ring N-atom of nicotinic acid benzyl ester can be alkylated, for instance, with 4-methoxyphenacyl bromide to provide 1-(4-methoxyphenyacyl)nicotinic acid benzyl ester bromide (Br. Patent 817103).

Compounds wherein Y is CH(R⁵)—C(O)—R⁷, wherein —C(O)—R⁷ comprises a carboxamide moiety, can be synthesized according to the method depicted in Scheme 5. An appropriately substituted amine can be condensed with an activated acetyl analog, containing a leaving group alpha to the carbonyl group (for example, an acid chloride such as α-chloroacetyl chloride), to provide a carboxamide. The carboxamide can then be used to alkylate the ring N atoms in the heterocycle to yield a compound of the invention. Alternatively, an activated acetyl analog with an α-halo leaving group can be used to directly alkylate the ring N-atom of the heterocycle. Displacement of the α-halo leaving group by an appropriately substituted amine also provides the N-alkylated heterocycle, wherein the —C(O)—R⁷ comprises a carboxamide.

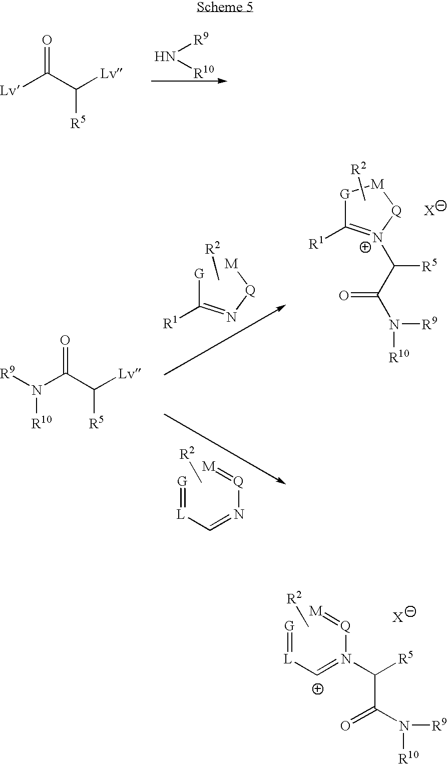

A useful synthetic route for the preparation of compounds of formula I wherein Y is —CH(R⁵)CN is shown in Scheme 6, wherein X is a halide, mesitylenesulfonate or other biologically acceptable anion. In Scheme 6, the appropriately substituted nitrogen containing aromatic heterocycle is contacted with an α halo substituted acetonitrile analog to produce cyanoalkyl substituted heterocycles. The reaction can be performed without any added solvent, or an anhydrous solvent can be utilized as the solvent medium. When a solvent is used, acetonitrile is a typical solvent for this reaction. Reaction times vary according to particular reactants and conditions, but are usually in the range of a few minutes to 48 hours at a temperature of 25-130° C.

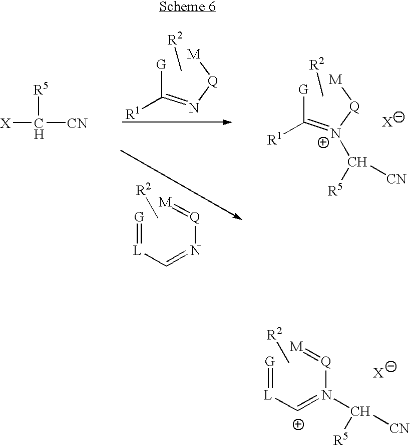

A synthetic scheme for making compounds of the formula I wherein Y is CH₂CH(OH)Rs is shown in Scheme 7. A hydroxyl is incorporated into a nucleophile used to derivatize a nitrogen heterocyclic compound, as follows:

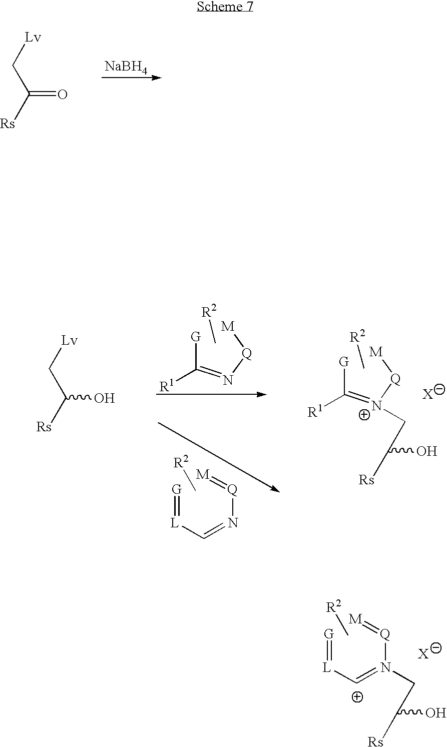

where Lv is a leaving group such as chloro. In a related synthesis, the carbonyl can be reduced with a stereoselective reducing agent such as (−) DIP-chloride [(−)-B-chlorodiisopinocampheylborane] or (+) DIP-chloride [(+)-B-chlorodiisopinocampheylborane] to provide specific stereoisomers of the alcohol. The alcohol can then be used to directly N-alkylate the heterocycle as above to prepare a compound of the invention enriched in the stereoisomer.

Compounds of the invention wherein R⁵ and R⁶ are both electron withdrawing groups such as ketones, carboxylic acids, carboxylic acid esters, carboxamides, or nitriles can be prepared as shown in Scheme 8. Suitable alkylating agents for compounds of this type include 2-halo substituted malonic acid derivatives such as 2-bromo diethyl malonate, 2-bromomalonamide, and the like. For instance, 1-bis(ethoxycarbonyl)-methylpyridinium bromide can be prepared from the reaction of 2-bromo diethyl malonate and pyridine in refluxing acetone (J. Chem Soc., Perkin Trans. I, 1981, 3059).

Likewise, 1-(2-malonamido)pyridinium bromide can be prepared from the reaction of 2-bromomalonamide and pyridine (U.S. Pat. No. 4,110,424).

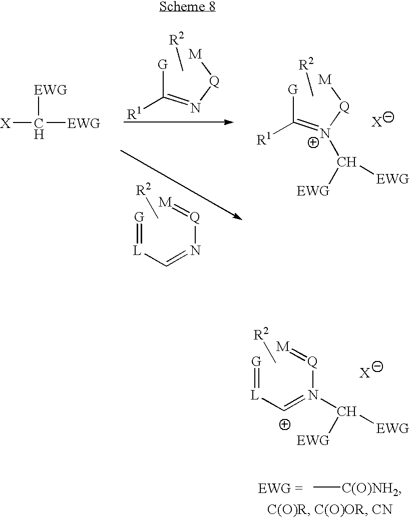

As is recognized in the art, many of the aromatic nitrogen heterocyclic analogs that serve as suitable precursors for the alkylation reactions discussed above are commercially available from chemical supply houses or are readily synthesized by methods well known in the art. For instance, certain substitution patterns can be obtained by electrophilic and nucleophilic substitution reactions on the heterocycle and are well known in the art. In addition, selected nitrogen heterocycles are susceptible to metallation with organoalkali reagents, for example, n-butyllithium. The intermediate lithio-heterocycles can be treated with electrophiles to provide additional routes to substituted aromatic nitrogen heterocyclic intermediates.

Certain aromatic nitrogen heterocyclic intermediates can be obtained by cyclization and cycloaddition reactions of substituted acyclic precursors that are well known in the art. Nonlimiting examples of the syntheses of nitrogen containing aromatic heterocyclic intermediates are described below.

Substituted pyrazoles can be obtained by reaction of 1,3-dicarbonyl compounds with hydrazines as was shown above in Scheme 2. As will be recognized by those in the art, use of unsymmetrically substituted 1,3-dicarbonyl compounds with alkyl or aryl hydrazines often lead to isomeric mixtures of pyrazole products. These isomeric mixtures can be separated by well-known separation techniques such as fractional crystallization, column chromatography, and the like. In addition, substituted pyrazole intermediates can be obtained by reaction of alkynyl carbonyls with hydrazines (Scheme 9) (Kost, A. N.; Grandberg Adv. Heterocyl. Chem., 1966, 6).

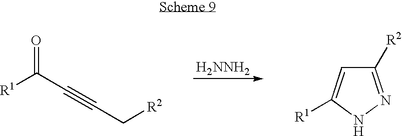

Substituted (1,2,3)triazole intermediates can be obtained by 1,3-dipolar cycloaddition reactions with activated alkynes (Scheme 10). For example, an alkyne diester can react with an azide to provide triazoles substituted at the 4 and 5 positions by ethoxycarbonyl groups, which serve as convenient moieties for further derivatization.

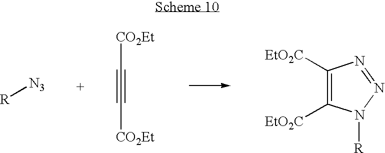

Benzotriazoles can be prepared, for example, by reaction of substituted ortho diaminobenzenes with nitrous acid (Scheme 11).

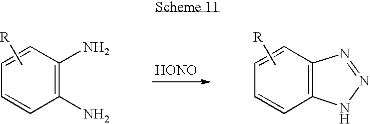

(1,2,4)Triazoles substituted in the 3 or 5 positions can be obtained from the condensation of acid hydrazides and thionoamides (Scheme 12). The triazole intermediates can be sequentially alkylated by two alkylating agents to provide compounds of the invention.

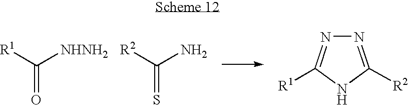

3- and 5-Aryl and alkyl isoxazoles can be prepared by reaction of the chloro substituted α,β-unsaturated ketones with hydroxylamine (Scheme 13). The isomeric products can be isolated by separation techniques such as fractional crystallization, distillation, or column chromatography. Alternatively, 5-aryl substituted isoxazoles can be prepared from acetophenones (Scheme 13, Lin, Y. Lang, S. A. J. Heterocyclic Chem., 1977, 14, 355).

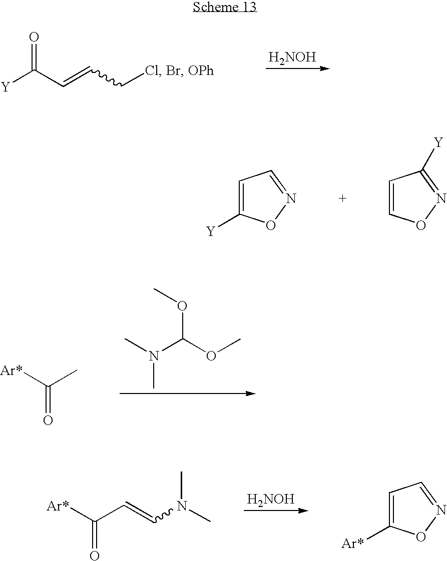

Alkyl and aryl substituted isothiazoles intermediates are prepared by the cyclization of β-imino thionocarbonyl compounds (Scheme 14). The cyclization is effected by oxidizing reagents well known in the art such as peroxides, chloranil, iodine, and the like. For example, starting material with an aryl thionocarbonyl group β-substituted to an imino group can be used to prepare a 5-aryl substituted isothiazole.

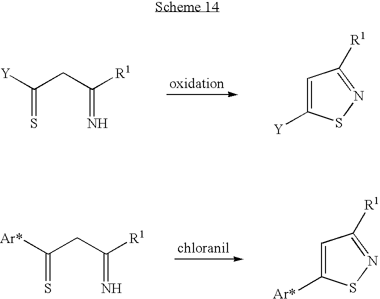

Suitable six-membered aromatic nitrogen heterocyclic intermediate such as pyrimidine, pyridazine, and pyridine can be obtained by ring cyclization and cycloaddition of substituted acyclic precursors as well. These heterocyclic intermediates serve as suitable substrates for the alkylation reactions discussed above to prepare the compounds of the invention.

Substituted pyrimidines can be obtained, for example, by the condensation of alkyl and aryl amidines with 1,3-dicarbonyl compounds (Scheme 15) or α,β,-unsaturated carbonyl compounds such as 3-ethoxymethacrolein.

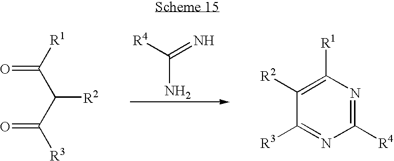

Benzo-fused pyrimidines (i.e., quinazolines) can be prepared, for instance, from benzene analogs containing an amino substituent ortho to a carbonyl (ketone or aldehyde) by acylation of the amino group with an alkanoyl or aroyl group, and then cyclization of the acylamino intermediate with ammonia (Scheme 16).

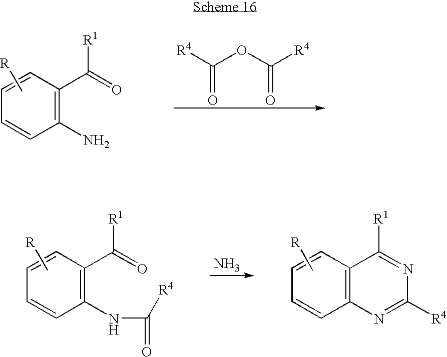

Pyridazines, useful as candidates for the alkylation reactions discussed above, can be prepared by reaction of hydrazine with 1,4-dicarbonyl compounds. The dihydro intermediates can be oxidized to give the desired pyridazines (Scheme 17). Phthalazines can be prepared in a similar fashion.

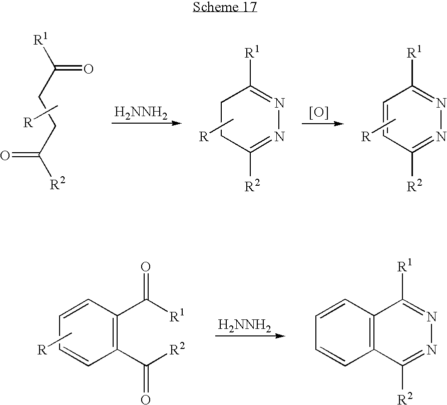

Cinnoline intermediates are prepared by cyclization of diazonium salts containing an ortho vinyl group (Scheme 18).

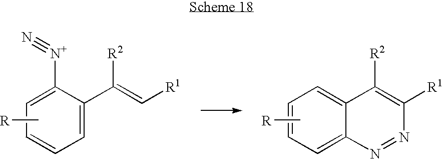

Quinolines that can serve as useful substrates for the alkylation reactions discussed above can be obtained from substituted benzene precursors by a number of methods known to those of ordinary skill in the art. For example, variations of the Skraup synthesis of quinolines can be used as shown in Scheme 19 (Jones, G., Quinolines, Wiley-Interscience, New York, 1977, p 93).

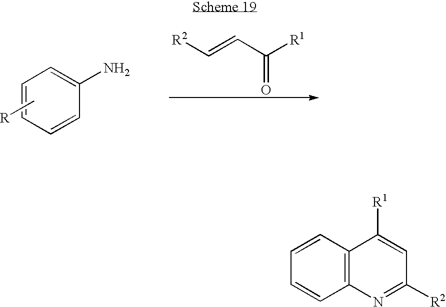

Substituted isoquinoline intermediates can be prepared by Bischler-Napieralski reaction followed by an oxidation step (Scheme 20).

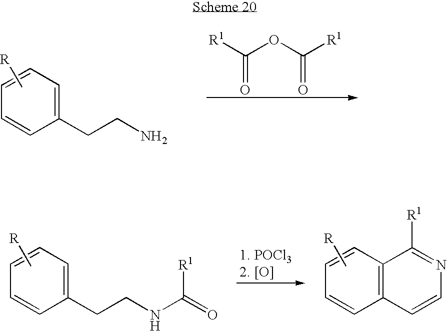

Pyridines, quinolines, and isoquinolines can be aminated with electrophilic aminating reagents such as hydroxylamine O-sulfonic acid (Scheme 21) or O-mesitylene sulfonylhydroxylamine.

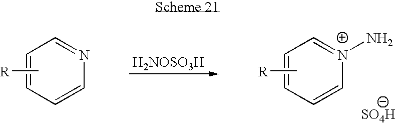

To treat the indications of the invention, an effective amount of a pharmaceutical compound will be recognized by clinicians but includes an amount effective to treat, reduce, ameliorate, eliminate or prevent one or more symptoms of the disease sought to be treated or the condition sought to be avoided or treated, or to otherwise produce a clinically recognizable change in the pathology of the disease or condition.

Pharmaceutical compositions can be prepared to allow a therapeutically effective quantity of the compound of the present invention, and can include a pharmaceutically acceptable carrier, selected from known materials utilized for this purpose. See, e.g., Remington, The Science and Practice of Pharmacy, 1995; Handbook of Pharmaceutical Excipients, 3^{rd} Edition, 1999. Such compositions can be prepared in a variety of forms, depending on the method of administration.

In addition to the subject compound, the compositions of this invention can contain a pharmaceutically-acceptable carrier. The term “pharmaceutically-acceptable carrier”, as used herein, means one or more compatible solid or liquid filler diluents or encapsulating substances that are suitable for administration to an animal, including a mammal or human. The term “compatible”, as used herein, means that the components of the composition are capable of being commingled with the subject compound, and with each other, such that there is no interaction that would substantially reduce the pharmaceutical efficacy of the composition under ordinary use. Preferably when liquid dose forms are used, the compounds of the invention are soluble in the components of the composition. Pharmaceutically-acceptable carriers must, of course, be of sufficiently high purity and sufficiently low toxicity to render them suitable for administration to the animal being treated.

Some examples of substances which can serve as pharmaceutically-acceptable carriers or components thereof are sugars, such as lactose, glucose and sucrose; starches, such as corn starch and-potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject compound is basically determined by the way the compound is to be administered. If the subject compound is to be injected, the preferred pharmaceutically-acceptable carrier is sterile, physiological saline, with a blood-compatible suspending agent, the pH of which has been adjusted to about 7.4.

If the preferred mode of administering the subject compound is perorally, the preferred unit dosage form is therefore tablets, capsules, lozenges, chewable tablets, and the like. Such unit dosage forms comprise a safe and effective amount of the subject compound, which is preferably from about 0.7 or 3.5 mg to about 280 mg/70 kg, more preferably from about 0.5 or 10 mg to about 210 mg/70 kg. The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder-mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of this invention, and can be readily made by a person skilled in the art.

Peroral compositions also include liquid solutions, emulsions, suspensions, and the like. The pharmaceutically-acceptable carriers suitable for preparation of such compositions are well known in the art. Such liquid oral compositions, preferably comprise from about 0.012% to about 0.933% of the subject compound, more preferably from about 0.033% to about 0.7%. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. Peroral liquid compositions may also contain one or more components such as sweeteners, flavoring agents and colorants disclosed above.

Other compositions useful for attaining systemic delivery of the subject compounds include sublingual and buccal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Compositions can also be used to deliver the compound to the site where activity is desired; such as eye drops, gels and creams for ocular disorders.

Compositions of this invention include solutions or emulsions, preferably aqueous solutions or emulsions comprising a safe and effective amount of a subject compound intended for topical intranasal administration. Such compositions preferably comprise from about 0.01% to about 10.0% w/v of a subject compound, more preferably from about 0.1% to about 2.0%. Similar compositions are preferred for systemic delivery of subject compounds by the intranasal route. Compositions intended to deliver the compound systemically by intranasal dosing preferably comprise similar amounts of a subject compound as are determined to be safe and effective by peroral or parenteral administration. Such compositions used for intranasal dosing also typically include safe and effective amounts of preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acids and bases to adjust the pH of these aqueous compositions as needed. The compositions may also comprise local anesthetics or other actives. These compositions can be used as sprays, mists, drops, and the like.

Other preferred compositions of this invention include aqueous solutions, suspensions, and dry powders comprising a safe and effective amount of a subject compound intended for atomization and inhalation administration. Such compositions are typically contained in a container with attached atomizing means. Such compositions also typically include propellants such as chlorofluorocarbons 12/11 and 12/114, and more environmentally friendly fluorocarbons, or other nontoxic volatiles; solvents such as water, glycerol and ethanol, these include cosolvents as needed to solvate or suspend the active; stabilizers such as ascorbic acid, sodium metabisulfite; preservatives such as cetylpyridinium chloride and benzalkonium chloride; tonicity adjustors such as sodium chloride; buffers; and flavoring agents such as sodium saccharin. Such compositions are useful for treating respiratory disorders, such as asthma and the like.

Other preferred compositions of this invention include aqueous solutions comprising a safe and effective amount of a subject compound intended for topical intraocular administration. Such compositions preferably comprise from about 0.01% to about 0.8% w/v of a subject compound, more preferably from about 0.05% to about 0.3%. Such compositions also typically include one or more of preservatives, such as benzalkonium chloride or thimerosal, vehicles, such as poloxamers, modified celluloses, povidone and purified water; tonicity adjustors, such as sodium chloride, mannitol and glycerin; buffers such as acetate, citrate, phosphate and borate; antioxidants such as sodium metabisulfite, butylated hydroxy toluene and acetyl cysteine; acids and bases can be used to adjust the pH of these formulations as needed.

Other preferred compositions of this invention useful for peroral administration include solids, such as tablets and capsules, and liquids, such as solutions, suspensions and emulsions (preferably in soft gelatin capsules), comprising a safe and effective amount of a subject compound. Such compositions can be coated by conventional methods, typically with pH or time-dependent coatings, such that the subject compound is released in the gastrointestinal tract at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit™ coatings, waxes and shellac.

The compounds of the invention are administered by ocular, oral, parenteral, including, for example, using formulations suitable as eye drops. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. See, Remington's Pharmaceutical Sciences, 16th Ed., Mack Publishing, Easton, Pa., 1980, as well as later editions, for information on pharmaceutical compounding.

Numerous additional administration vehicles will be apparent to those of ordinary skill in the art, including without limitation slow release formulations, liposomal formulations and polymeric matrices.

In another preferred embodiment, the pharmaceutically effective amount is approximately 0.1 or 0.5 to 4 mg/kg body weight daily (or 0.1 or 0.5 to 4 mg/kg daily, particularly with less active members of the genus). Still more preferably, the pharmaceutically effective amount is approximately 1 mg/kg body weight daily. In one embodiment, the amount is administered in once daily doses, each dose being approximately 1 mg/kg body weight. In another embodiment, the amount is administered in twice daily doses, with the daily dose being approximately 1 mg/kg body weight.

The activity of the compounds of the invention in breaking, reversing or inhibiting the formation of AGE's or AGE-mediated crosslinks can be assayed by any of the methods described in U.S. Pat. No. 5,853,703.

Except where heteroaryl is separately recited for the same substituent, the term “heterocycle” includes heteroaryl.

Where noted above, publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

### EXAMPLE 1

### 5-Amino-3-carbamoylmethyl-[1,3,4]-thiadiazolium Bromide

1 g 2-aminothiadiazole was reacted with 1.36 g 2-bromoacetamide in 10 g acetonitrile at reflux for 5 hours. On cooling to room temperature solids were filtered, washed with MTBE and dried. The yield was 1.9 g. The product was further purified by recrystallization from MeOH. The recrystallized compound had a melting point of 207-209° C. and proton NMR spectra consistent with the structure 5-amino-3carbamoylmethyl-[1,3,4]-thiadiazolium bromide. On analysis the compound contained 20.17% C, 2.73% H, 33.14% Br, 23.58% N, and 13.15% S.

### 2-Amino-3-(4-chloro-benzyl)-[1,3,4]-thiadiazolium Chloride

1 g 2-aminothiadiazole was reacted with 1.89 g 1-Chloro-4-chloromethylbenzene in 10 g acetonitrile at reflux for 5 hours. On cooling to room temperature solids were filtered, washed with MTBE and dried. The yield was 0.4 g. The product was further purified by recrystallization from MeOH. The recrystallized compound had a melting point of 226-227° C. and proton NMR spectra consistent with the structure 2-amino-3-(4-chloro-benzyl) chloride. On analysis the compound contained 39.49% C, 3.15% H, 25.24% Cl, 15.14% N, and 11.39% S.

### 2-Amino-3-(4-fluoro-benzyl)-[1,3,4]-thiadiazolium Bromide

1 g 2-aminothiadiazole was reacted with 1.87 g 1-fluoro-4-bromomethyl-benzene in 10 g acetonitrile at reflux for 5 hours. On cooling to room temperature solids were filtered, washed with MTBE and dried. The yield was 2.15 g. The product was further purified by recrystallization from MeOH. The recrystallized compound had a melting point of 236-238° C. and proton NMR spectra consistent with the structure 2-amino-3-(4-fluro-benzyl)-[1,3,4]-thiadiazolium bromide. NMR analysis was possibly consistent with the 5-amino compound, though both isomers are believed useful in the invention. On analysis the compound contained 37.30% C, 2.88% H, 27.35% Br, 14.45% N, and 11.0% S.

### EXAMPLE 2

### 3-(Aminocarbonyl)-1-[2-(4-chlorophenyl)-2-oxoethyl]pyridinium Chloride

1.22 g nicotinamide was reacted with 2.27 g 2-chloro-1-(4-chlorophenyl)ethanone in 10 g acetonitrile at reflux for 5 hours. On cooling to room temperature, solids were filtered and dried. The yield was 2.2 g. The material was dissolved in methanol and recrystallized with MTBE. The recrystallized compound had a melting point of 264-265° C. and proton NMR spectra consistent with the structure 3-(aminocarbonyl)-1-[2-(4-chlorophenyl)-2-oxoethyl]pyridinium chloride. On analysis the compound contained 54.03% C, 3.75% H, 22.96% Cl, and 9.00% N.

### 3-(Aminocarbonyl)-1-benzylpyridinium Bromide

1.0 g nicotinamide was reacted with 1.4 g (bromomethyl)benzene at reflux for 4 hours. On cooling to room temperature, solids were filtered and dried. The yield was 2.26 g. The material was dissolved in methanol and recrystallized with MTBE. The recrystallized compound had a melting point of 210-212° C. and proton NMR spectra consistent with the structure 3-(aminocarbonyl)-1-benzylpyridinium bromide. On analysis the compound contained 53.23% C, 4.42% H, 27.95% Br, and 9.49% N.

### 3-Carbamoyl-1-(4-methoxy-benzyl)-pyridinium Chloride

1 g Nicotinamide was reacted with 1.28 g 1-chloromethyl-4-methoxy-benzene in 20 ml acetonitrile at reflux for 5 hours. On cooling to room temperature solids were filtered, washed with MTBE and dried. The yield was 1.32 g. The compound had a melting point of 233-234° C. and proton NMR spectra consistent with the structure 3-carbamoyl-1-(4-methoxy-benzyl)-pyridinium chloride. On analysis the compound contained 60.32% C, 5.49% H, 12.97% Cl, and 10.06% N.

### 3-Carbamoyl-1-[2-(4-fluoro-phenyl)-2-oxo-ethyl]-pyridinium Chloride

1 g Nicotinamide was reacted with 1.28 g 2-chloro-1-(4-fluoro-phenyl)-ethanone in 20 ml acetonitrile at reflux for 5 hours. On cooling to room temperature solids were filtered, washed with MTBE and dried. The yield was 0.79 g. The compound had a melting point of 221-225° C. and proton NMR spectra consistent with the structure 3-carbamoyl-1-[2-(4-fluoro-phenyl)-2-oxo-ethyl]-pyridinium chloride. On analysis the compound contained 56.88% C, 4.00% H, 12.18% Cl, 6.19% F, and 9.46% N.

### EXAMPLE 3

### 1-(4-Fluoro-benzyl)-pyrimidin-1-ium Bromide

Pyrimidine (0.55 g) was reacted with 1.3 g 1-bromomethyl-4-fluoro-benzene in 20 ml acetonitrile at reflux for 5 hours. On cooling to room temperature solids were filtered, washed with MTBE and dried. The yield was 1.23 g. The product was further purified by dissolving in MeOH and precipitating with MTBE. The recrystallized compound had a melting point of 230-231 ° C. and proton NMR spectra consistent with the structure 1-(4-fluoro-benzyl)-pyrimidin-1-ium bromide. On analysis the compound contained 48.98% C, 3.71% H, 29.56% Br, 7.08% F, and 10.4% N.

### 1-(4-Chloro-benzyl)-pyrimidin-1-ium Chloride

1 g pyrimidine was reacted with 2.01 g 1-chloromethyl-4-bromo-benzene in 20 ml CH₃CN acetonitrile at for 5 hours. On cooling to room temperature solids were filtered, washed with MTBE and dried. The yield was 0.4 g. The product was further purified by dissolving in EtOH and precipitating with MTBE. The recrystallized compound had a melting point of 190-191° C. and proton NMR spectra consistent with the structure 1-(4-chloro-benzyl)-pyrimidin-1-ium chloride. On analysis the compound contained 54.30% C, 4.21% H, 29.14% Cl, and 11.40% N.

### EXAMPLE 4

Rats received a daily intraperitoneal dose of 10 mg/kg 3-[2-phenyl-2-oxoethyl]-4,5-dimethyl-thiazolium salt (compound A) (n=14) or placebo (n=15) for 30 days. The animals then underwent a thoracotomy and the left anterior descending coronary artery ligated. The chest was then closed and the animals allowed to recover for 14 days while continuing to be treated with compound A or placebo. The animals were then sacrificed and the hearts removed for histological examination. The weight of the infarcted tissue was 0.16±0.04 g for the placebo treated animals compared to 0.11±0.05 g for the compound A treated animals (p=0.04). The thickness of the ventricular wall in the infarcted zone was also reduced in the compound A treated animals compared to placebo (2.72±0.13 mm vs. 2.56±0.22 mm, p=0.09).

### EXAMPLE 5

### Cross-linking Inhibition Assay

The following method was used to evaluate the ability of the compounds to inhibit the cross-linking of glycated bovine serum albumin (AGE-BSA) to rat tail tendon collagen-coated 96-well plates.

AGE-BSA was prepared by incubating BSA at a concentration of 200 mg per ml with 200 mM glucose in 0.4M sodium phosphate buffer, pH 7.4 at 37° C. for 12 weeks. The glycated BSA was then extensively dialyzed against phosphate buffer solution (PBS) for 48 hours with additional 5 times buffer exchanges. The rat tail tendon collagen coated plate was blocked first with 300 microliters of Superbloc blocking buffer (Pierce Chemical, Rockford, Ill.) for one hour. The blocking solution was removed from the wells by washing the plate twice with phosphate buffered saline (PBS)-Tween 20 solution (0.05% Tween 20) using a NUNC-multiprobe (Nalge Nunc, Rochester, N.Y.) or Dynatech ELISA-plate (Dynatech, Alexandria, Va.) washer. Cross-linking of AGE-BSA (1 to 10 microgram per well depending on the batch of AGE-BSA) to rat tail tendon collagen coated plate was performed with and without the testing compound dissolved in PBS buffer at pH 7.4 at one or more desired concentrations by the addition of 50 microliters each of the AGE-BSA diluted in PBS or in the solution of test compound at 37° C. for 4 hours. Unbrowned BSA in PBS buffer with or without testing compound were added to the separate wells as the blanks. The un-cross-linked AGE-BSA was then removed by washing the wells three times with PBS-Tween buffer. The amount of AGE-BSA crosslinked to the tail tendon collagen-coated plate was then quantitated using a polyclonal antibody raised against AGE-RNase. After a one-hour incubation period, AGE antibody was removed by washing 4 times with PBS-Tween.

The bound AGE antibody was then detected with the addition of horseradish peroxidase-conjugated secondary antibody-goat anti-rabbit immunoglobulin and incubation for 30 minutes. The substrate of 2,2-azino-di(3-ethylbenzthiazoline sulfonic acid) (ABTS chromogen) (Zymed Laboratories, Inc., South San Francisco, Calif.) was added. The reaction was allowed for an additional 15 minutes and the absorbance was read at 410 nm in a Dynatech plate reader.

### EXAMPLE 6

### Cross-link Breaking Assay

To ascertain the ability of the compounds of the instant invention to break or reverse already formed advanced glycosylation endproducts, a sandwich enzyme immunoassay was applied. Generally, the assay utilizes collagen-coated 96 well microtiter plates that are obtained commercially. AGE-modified protein (AGE-BSA) is incubated on the collagen-coated wells for four hours, is washed off the wells with PBS-Tween and solutions of the test compounds are added. Following an incubation period of 16 hours (37° C.) cross-link-breaking is detected using an antibody raised against AGE-ribonuclease or with an antibody against BSA.

Preparation of Solutions and Buffers

Bovine Serum Albumin (Type V) (BSA) (from Calbiochem) solution was prepared as follows: 400 mg of Type V BSA (bovine serum albumin) was added for each ml of 0.4 M sodium phosphate buffer, pH 7.4. A 400 mM glucose solution was prepared by dissolving 7.2 grams of dextrose in 100 ml of 0.4 M sodium phosphate buffer, pH 7.4. The BSA and glucose solutions were mixed 1:1 and incubated at 37° C. for 12 weeks. The pH of the incubation mixture was monitored weekly and adjusted to pH 7.4 if necessary. After 12 weeks, the AGE-BSA solution was dialyzed against PBS for 48 hours with four buffer changes, each at a 1:500 ratio of solution to dialysis buffer. Protein concentration was determined by the micro-Lowry method. The AGE-BSA stock solution was aliquoted and stored at −20° C.

Test compounds were dissolved in PBS and the pH was adjusted to pH 7.4, if necessary. AGE-BSA stock solution was diluted in PBS to measure maximum crosslinking and in the inhibitor solution for testing inhibitory activity of compounds. The concentration of AGE-BSA necessary to achieve the optimum sensitivity was determined by initial titration of each lot of AGE-BSA.

Substrates for detection of secondary antibody binding were prepared by diluting the HRP substrate buffer (Zymed) 1:10 in distilled water and mixing with ABTS chromogen (Zymed) 1:50 just prior to use.

Assay Procedures

Biocoat plates were blocked with 300 microliters of Superbloc (Pierce Chemical). Plates were blocked for one hour at room temperature and were washed with PBS-Tween (0.05% v/v) three times with the Dynatech platewasher before addition of test reagents.

The first three wells of the Biocoat plate were used for the reagent blank. Fifty microliters of solutions AGE-BSA were added to test wells in triplicate and only PBS in blank wells. The plate was incubated at 37° C. for four hours and washed with PBS-Tween three times. Fifty microliters of PBS was added to the control wells and 50 microliters of the test prospective agent was added to the test wells and blank. The plate was incubated overnight (approximately 16 hours) with prospective agent, followed by washing in PBS before addition of primary antibody.

(Prior to use, each lot of primary antibody, either anti-BSA or anti-RNase, was tested for optimum binding capacity in this assay by preparing serial dilutions (1:500 to 1:2000) and plating 50 microliters of each dilution in the wells of Biocoat plates. Optimum primary antibody was determined from saturation kinetics.) Fifty microliters of primary antibody of appropriate dilution, was added and incubated for one hour at room temperature. The plate was then washed with PBS-Tween.

Plates were incubated with the secondary antibody, HRP-(Goat-anti-rabbit), which was diluted 1:4000 in PBS and used as the final secondary antibody. The incubation was performed at room temperature for thirty minutes.

Detection of maximum crosslinking and breaking of AGE crosslinking was performed as follows. HRP substrate (100 microliter) was added to each well of the plate and was incubated at 37° C. for fifteen minutes. Readings were taken in the Dynatech ELISA-plate reader.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of treating or ameliorating an indication of the invention in an animal, including a human, comprising administering an effective amount of (A) a compound of formula (I):
Y—Ar^{⊕}.X⁻ (I)
wherein:
a. Ar is a five or six membered heteroaryl ring having a first ring nitrogen and optionally second or third ring nitrogens, with the remaining ring atoms being carbon, oxygen, or sulfur, provided the first nitrogen of Ar is a quaternary nitrogen and Ar is not thiazolium, oxazolium or imidazolium;
b. Y is substituted on the first ring nitrogen, with the proviso that if Ar is pyrazole, indazole, (1,2,3)-triazole, benzotriazole, or (1,2,4)-triazole, the second ring nitrogen is substituted with
1. alkyl or alkoxycarbonylalkylene;
2. Ar* {wherein, consistent with the rules of aromaticity, Ar* is C₆ or C₁₀ aryl or a 5- or 6-membered heteroaryl ring, wherein 6-membered heteroaryl ring contains one to three atoms of N, and the 5-membered heteroaryl ring contains from one to three atoms of N or one atom of O or S and zero to two atoms of N, each heteroaryl ring may be fused to a benzene, pyridine, pyrimidine, pyridazine, pyrazine, or (1,2,3)triazine (wherein the ring fusion is at a carbon-carbon double bond of Ar*)}; or
3. Ar*alkyl-, Ar*C(O)alkyl-, Ar*sulfonylalkyl-, or Ar*sulfinylalkyl-; and
c. Ar can be substituted on ring carbon atoms
1. with one or more substituents independently selected from the group consisting ω-alkylenesulfonic acid, carbamoyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or
2. two adjacent substitutions together with their ring carbons form a C₆- or C₁₀-aromatic fused ring system; or
3. two adjacent substitutions together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo; or
4. two adjacent substitutions together with their ring carbons form a fused five to eight membered heterocycle, wherein the ring fusion is at a carbon-carbon double bond of Ar, wherein the heterocycle consists of ring atoms selected from the group consisting of carbon, nitrogen, oxygen, and S(O)ₙ, wherein n=0,1, or 2; or
5. two adjacent substitutions together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur;
d. Y is:
1. a group of the formula —CH(R⁵)—R⁶
(a) R⁵ is hydrogen, alkyl-, cycloalkyl-, alkenyl-, alkynyl-, aminoalkyl-, hydroxy[C₁ to C₆]alkyl, dialkylaminoalkyl-, (N—[C₆ or C₁₀]aryl)(N-alkyl)aminoalkyl-, piperidin-1-ylalkyl-, pyrrolidin-1-ylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperazin-1-ylalkyl, piperidin-1-ylalkyl, [C₆ or C₁₀]aryl, or independently the same as R⁶;
(b) wherein R⁶ is
(1) hydrogen, alkyl (which may be substituted by alkoxycarbonyl)-, alkenyl, alkynyl, cyano-, cyanoalkyl-, or Rs, wherein Rs is a [C₆ or C₁₀]aryl or a heterocycle containing 4-10 ring atoms of which 1-3 are heteroatoms selected from the group consisting of oxygen, nitrogen and sulfur; or
(2) a group of the formula —W—R⁷, wherein R⁷ is alkyl, alkoxy, hydroxy, or Rs, wherein W is —C(═O)— or —S(O)₂—;
(3) a group of the formula —W—OR⁸ wherein R⁸ is hydrogen or alkyl,
(4) a group of the formula —CH(OH)Rs; or
(5) a group of the formula —W—N(R⁹)R¹⁰, wherein
(a) R⁹ is hydrogen and R¹⁰ is an alkyl or cycloalkyl, optionally substituted by
(i) [C₆ or C₁₀]aryl, or
(ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C1-C3)alkylenedioxy groups, or fused to a phenyl or pyridine ring, wherein the ring fusion is at a carbon-carbon double bond of the heteroaryl ring), or
(iii) a heterocycle containing 4-10 ring atoms of which 1-3 are heteroatoms selected from the group consisting of oxygen, nitrogen and sulfur; or
(b) R⁹ is hydrogen or alkyl and R¹⁰ is Ar*; or
(c) R⁹ is hydrogen or alkyl, R¹⁰ is a heterocycle containing 4-10 ring atoms of which 1-3 are heteroatoms are selected from the group consisting of oxygen, nitrogen and sulfur; or
(d) R⁹ and R¹⁰ are both alkyl groups; or
(e) R⁹ and R¹⁰ together with N form a heterocycle containing 4-10 ring atoms which can incorporate up to one additional heteroatom selected from the group of N, O or S in the ring, wherein the heterocycle is optionally substituted with (C₆- or C₁₀)aryl, (C₆- or C₁₀)arylalkyl, or a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy; or ∝(f) R⁹ and R¹⁰ are both hydrogen; or
2. —NH₂, and
e. X is a pharmaceutically acceptable anion, which may be absent if the compound provides a neutralizing salt,
(B) a pharmaceutically acceptable salt of the compound,
wherein aryl, Ar or Ar* can be substituted with, in addition to any substitutions specifically noted, one or more general substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*C(O)NH—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO₃H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, thiomorpholin-4-yl, piperidin-1-yl; and
wherein heterocycles, except those of Ar or Ar*, can be substituted with, in addition to any substitutions specifically noted, the following general substitutions: acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*—, carboxy, dialkylamino, fluoro, fluoroalkyl, difluoroalkyl, hydroxy, mercapto, sulfamoyl, or trifluoromethyl.

2. The method of claim 1, comprising administering an effective amount of a compound of formula I, wherein Y is according to formula —CH(R⁵)R⁶.

3. The method of claim 2, comprising administering an effective amount of a compound of formula I, wherein Y is according to formula —CH(R⁵)—W—R⁷.

4. The method of claim 2, comprising administering an effective amount of a compound of formula I, wherein Y is according to formula —CH(R⁵)—W—Rs.

5. The method of claim 1, comprising administering an effective amount of a compound of formula I, wherein:
c. Ar can substituted on ring carbon atoms
1. with one or more substituents independently selected from the group consisting hydrogen, acylamino, alkanoyl, alkanoylalkyl, alkoxy, alkoxycarbonyl, alkoxycarbonylalkyl, alkyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid (—SO₃H), alkylsulfonyl (alkylSO₂—), alkylsulfinyl (alkylSO—), alkylthio, trifluoromethyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—, wherein Ar* may be substituted by one or more substituents as set forth above; or
2. two adjacent substitutions together with their ring carbons form a C₆- or C₁₀-aromatic fused ring system; or
3. two adjacent substitutions together with their ring carbons form a C₅-C₇ fused cycloalkyl ring having no double bonds except the fused double bond of the Ar group, which cycloalkyl ring can be substituted by one or more of the group consisting of alkyl, amino, aminocarbonyl, carboxy, fluoro, or oxo, wherein multiple substituents are located on different carbon atoms of the cycloalkyl ring, except in the case of alkyl, and fluoro substituents, which can be located on the same or different carbon atoms;
d. Y is:
1. a group of the formula —CH(R⁵)—R⁶
(a) R⁵ is hydrogen or alkyl;
(b) wherein R⁶ is
(1) hydrogen, alkyl , alkenyl, alkynyl, cyano, cyanoalkyl, or Rs, wherein Rs is a [C₆ or C₁₀]aryl or a heterocycle containing 4-10 ring atoms; or
(2) a group of the formula —W—R⁷, wherein R⁷ is alkyl, alkoxy, hydroxy, or Rs, wherein W is —C(═O)— or —S(O)₂—;
(3) a group of the formula —W—OR⁸ wherein R⁸ is hydrogen or alkyl,
(4) a group of the formula —CH(OH)Rs; or
(5) a group of the formula —W—N(R⁹)R¹⁰, wherein
(a) R⁹ is hydrogen and R¹⁰ is an alkyl or cycloalkyl, optionally substituted by
(i) [C₆ or C₁₀]aryl, or
(ii) a 5- or 6-membered heteroaryl ring that, in addition to the general substitutions, can be optionally substituted with one or more halo or (C₁-C₃)alkylenedioxy groups, or fused to a phenyl ring, or
(b) R⁹ is hydrogen or alkyl and R¹⁰ is Ar*; or
(e) R⁹ and R¹⁰ together with N form a heterocycle containing 4-10 ring atoms which can incorporate up to one additional heteroatom selected from the group of N, O or S in the ring, wherein the heterocycle is optionally substituted with (C₆- or C₁₀)aryl, (C₆-or C₁₀)arylalkyl, or a 5- or 6-membered heteroaryl ring containing at least one and up to three atoms of N for the 6-membered heteroaryl rings and from one to three atoms of N or one atom of 0 or S and zero to two atoms of N for the 5-membered heteroaryl rings, each such heteroaryl can be optionally substituted with one or more halo or (C₁-C₃)alkylenedioxy; or
(f) R⁹ and R¹⁰ are both hydrogen;
or
2. —NH₂, and
e. X is a pharmaceutically acceptable anion, which may be absent if the compound provides a neutralizing salt,
(B) a pharmaceutically acceptable salt of the compound,
wherein aryl, Ar or Ar* can be substituted with, in addition to any substitutions specifically noted, with one or more substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar*C(O)—, Ar*C(O)NH—, Ar*O—, Ar*—, Ar*-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid (SO₃H); and
wherein heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylsulfonyl, alkylsulfinyl, alkylthio, Ar*C(O)—, Ar*O—, Ar*—, carboxy, fluoro, fluoroalkyl, difluoroalkyl, hydroxy, mercapto, sulfamoyl, or trifluoromethyl, wherein multiple substituents are located on different atoms of the heterocyclic ring , with the proviso that alkyl, alkylcarbonyl, and fluoro substituents can be substituted on the same carbon atom of the heterocyclic ring.

6. The method of claim 5, comprising administering an effective amount of a compound of formula I, wherein Y is according to formula —CH(R⁵)R⁶.

7. The method of claim 6, comprising administering an effective amount of a compound of formula I, wherein Y is according to formula —CH(R⁵)—W—R⁷.

8. The method of claim 6, comprising administering an effective amount of a compound of formula I, wherein Y is according to formula —CH(R⁵)—W—Rs.

9. The method of claim 1, wherein Y—Ar^{⊕}.X⁻ is 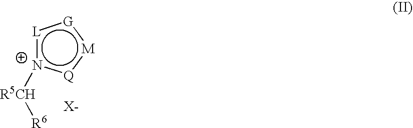
wherein G, L, M, and Q are independently O, S, N, N—R^{a}, C, C—R^{b}, C—R^{c}, C—R, wherein no more than one of G, L, M, or Q is O or S;
wherein
1. R⁵ is H;
2. R⁶ is
(1) cyano or
(2) a group of the formula —W—R⁷, wherein R⁷ is alkyl or Rs, and W is C(═O)— or —S(═O)—;
(3) a group of the formula —W—N(R⁹)R¹⁰, wherein
(a) R⁹ is hydrogen and R¹⁰ is an alkyl or cycloalkyl, optionally substituted by
(i) [C₆ or C₁₀]aryl, or
(ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, piperidin-1-yl, halo or (C₁-C₃)alkylenedioxy groups, or fused to a phenyl or pyridine ring, wherein the ring fusion is at a carbon-carbon double bond of the heteroaryl ring);
3. R^{a} is alkyl, Ar*, Ar*alkyl, alkoxycarbonylalkylene-, Ar*C(O)alkyl-, Ar*sulfonylalkyl-, or Ar*sulfinylalkyl-; and
4. R^{b}, R^{c}, and R^{d} are
(a) independently selected from the group consisting hydrogen, acylamino, acyloxyalkyl, alkanoyl, alkanoylalkyl, alkenyl, alkoxy, alkoxycarbonyl, alkoxycarbonylalkyl, alkyl, alkylamino, (C1-C3)alkylenedioxy, alkylsulfonyl, alkylsulfinyl, ω-alkylenesulfonic acid, alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO₃H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, piperidin-1-yl;
(b) wherein any two of R^{b}, R^{c}, and R^{d} are adjacent, together with their ring carbons form a C₆ or C₁₀ aromatic fused ring system;
(c) wherein any two of R^{b}, R^{c}, and R^{d} are adjacent, together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo;
(d) wherein any two of R^{b}, R^{c}, and R^{d} are adjacent, together with their ring carbons form a fused five to eight membered heterocycle, wherein the ring fusion is at a carbon-carbon double bond of Ar, wherein the fused heterocycle consists of ring atoms selected from the group consisting of carbon, nitrogen, oxygen, and S(O)ₙ wherein n=0,1, or 2; and
(e) wherein any two of R^{b}, R^{c}, and R^{d} are adjacent, together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur; and

10. The method of claim 9, wherein Ar is not tetrazole or pyrrole.

11. The method of claim 9, comprising administering an effective amount of a compound of formula II, wherein R⁶ is according to —CH(R⁵)—W—Rs

12. The method of claim 9, wherein aryl, Ar or Ar* is substituted with, in addition to any substitutions specifically noted, one or more substituents selected from the group consisting of hydrogen, alkyl, amino, dialkylamino, 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, piperidin-1-yl.

13. The method of claim 9, wherein Y—Ar^{⊕}.X⁻ is 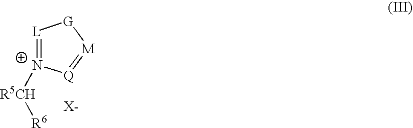
wherein G is O, S, or N—R^{a};
M is N or C—R^{b};
Q is N or C—R^{c}; and
L is N or C—R^{d}.

14. The method of claim 9, wherein Y—Ar^{⊕}.X⁻ is 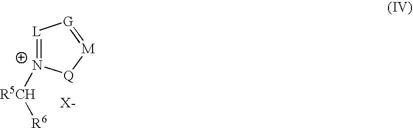
wherein G is N or C—R^{c};
M is N or C—R^{b};
Q is O, S, or N—R^{a}; and
L is N or C—R^{d}.

15. The method of claim 9, wherein Y—Ar^{⊕}.X⁻ is

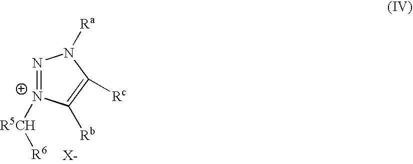

16. The method of claim 1, wherein Y—Ar^{⊕}.X⁻ is 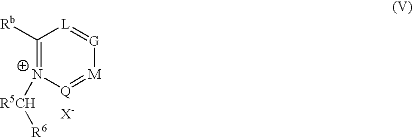
wherein L, G, M, Q, or R are independently N, C—R^{c}, C—R^{d}, C—R^{e}, C—R^{f},
wherein
1. R⁵ is H;
2. R⁶ is
(1) cyano or
(2) a group of the formula —W—R⁷, wherein R⁷ is alkyl or Rs, and W is C(═O)— or —S(═O)—;
3. R^{b}, R^{e}, R^{d}, and R^{e} are
(a) independently selected from the group consisting hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO₃H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, piperidin-1-yl;
(b) where any two of R^{b}, R^{c}, R^{d}, and R^{e} are adjacent, together with their ring carbons form a C₆- or C₁₀-aromatic fused ring system;
(c) where any two of R^{b}, R^{c}, R^{d}, and R^{e} are adjacent, together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo;
(d) wherein any two of R^{b}, R^{c}, R^{d}, and R^{e} are adjacent, together with their ring carbons form a fused five to eight membered heterocycle, wherein the ring fusion is at a carbon-carbon double bond of Ar, wherein the fused heterocycle consists of ring atoms selected from the group consisting of carbon, nitrogen, oxygen, and S(O)ₙ wherein n=0,1, or 2;
(e) wherein any two of R^{b}, R^{c}, R^{d}, and R^{e} are adjacent, together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur, and
wherein Ar has no more than three nitrogen atoms in the ring.

17. The method of claim 1, wherein Ar is substituted on a said ring nitrogen with amino.

18. The method of claim 17, wherein Ar is further substituted with up to two aminos.

19. The method of claim 1, wherein:
a. Ar is a five or six membered heteroaryl ring having a first ring nitrogen and optionally second or third ring nitrogens, with the remaining ring atoms being carbon, oxygen, or sulfur, provided the first nitrogen of Ar is a quaternary nitrogen and Ar is not thiazolium, oxazolium or imidazolium;
b. Y is substituted on the first ring nitrogen, with the proviso that if Ar is pyrazole, indazole, (1,2,3)-triazole, benzotriazole, or (1,2,4)-triazole, the second ring nitrogen is substituted with
1. alkyl or alkoxycarbonylalkylene;
2. Ar*; or
3. Ar*alkyl-, Ar*C(O)alkyl-, Ar*sulfonylalkyl-, or Ar*sulfinylalkyl-; and
c. Ar can be substituted on ring carbon atoms
1. with one or more substituents independently selected from the group consisting ω-alkylenesulfonic acid, carbamoyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or
2. two adjacent substitutions together with their ring carbons form a C₆- or C₁₀-aromatic fused ring system; or
3. two adjacent substitutions together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, aminocarbonyl, carboxy, fluoro, or oxo; or
4. two adjacent substitutions together with their ring carbons form a fused five to eight membered heterocycle, wherein the ring fusion is at a carbon-carbon double bond of Ar, wherein the heterocycle consists of ring atoms selected from the group consisting of carbon, nitrogen, oxygen, and S(O)ₙ, wherein n=0,1, or 2; or
5. two adjacent substitutions together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur;
d. Y is:
1. a group of the formula —CH(R⁵)—R⁶
(a) R⁵ is hydrogen, alkyl-, cycloalkyl-, alkenyl-, alkynyl-, hydroxy[C₁ to C₆]alkyl, [C₆ or C₁₀]aryl, or independently the same as R⁶;
(b) wherein R⁶ is
(1) hydrogen, alkyl (which may be substituted by alkoxycarbonyl)-, alkenyl, alkynyl, cyano-, cyanoalkyl-, or Rs, wherein Rs is a [C₆ or C₁₀]aryl or a heterocycle containing 4-10 ring atoms of which 1-3 are heteroatoms selected from the group consisting of oxygen, nitrogen and sulfur; or
(2) a group of the formula —W—R⁷, wherein R⁷ is alkyl, alkoxy, hydroxy, or Rs, wherein W is —C(═O)— or —S(O)₂—;
(3) a group of the formula —W—OR⁸ wherein R⁸ is hydrogen or alkyl,
(4) a group of the formula —CH(OH)Rs; or
(5) a group of the formula —W—N(R⁹)R¹⁰, wherein
(a) R⁹ is hydrogen and R¹⁰ is an alkyl or cycloalkyl, optionally substituted by
(i) [C₆ or C₁₀]aryl, or
(ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more halo or (C1-C3)alkylenedioxy groups, or fused to a phenyl, or
(iii) a heterocycle containing 4-10 ring atoms of which 1-3 are heteroatoms selected from the group consisting of oxygen, nitrogen and sulfur; or
(b) R⁹ is hydrogen or alkyl and R¹⁰ is Ar*; or
(c) R⁹ is hydrogen or alkyl, R¹⁰ is a heterocycle containing 4-10 ring atoms of which 1-3 are heteroatoms are selected from the group consisting of oxygen, nitrogen and sulfur; or
(d) R⁹ and R¹⁰ are both alkyl groups; or
(e) R⁹ and R¹⁰ together with N form a heterocycle containing 4-10 ring atoms which can incorporate up to one additional heteroatom selected from the group of N, O or S in the ring, wherein the heterocycle is optionally substituted with (C₆- or C₁₀)aryl, (C₆- or C₁₀)arylalkyl, or a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings, each such heteroaryl can be optionally substituted with one or more halo or (C₁-C₃)alkylenedioxy; or
(f) R⁹ and R¹⁰ are both hydrogen; or
2. —NH₂, and
e. X is a pharmaceutically acceptable anion, which may be absent if the compound provides a neutralizing salt,
(B) a pharmaceutically acceptable salt of the compound,
wherein aryl, Ar or Ar* can be substituted with, in addition to any substitutions specifically noted, one or more substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar*C(O)—, Ar*C(O)NH—, Ar*O—, Ar*—, Ar*-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid (SO₃H); and
wherein heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylsulfonyl, alkylsulfinyl, alkylthio, Ar*C(O)—, Ar*O—, Ar*—, carboxy, fluoro, fluoroalkyl, difluoroalkyl, hydroxy, mercapto, sulfamoyl, or trifluoromethyl.

20. A compound of formula VI: 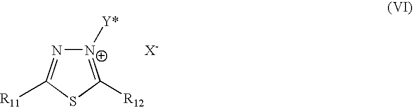
wherein
a. one of R¹¹ and R¹² is hydrogen, and the other is selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1 yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar² (wherein Ar² is C₆ or C₁₀ aryl), Ar²alkyl, Ar²—O, Ar²SO₂—, Ar²SO—, Ar²S—, Ar²SO₂NH—, Ar²NH, (N—Ar²)(N-alkyl)N—, Ar²C(O)—, Ar²C(O)NH—, Ar²NH—C(O)—, or (N—Ar²)(N-alkyl)N—C(O)—;
b. Y* is a group of the formula —CH(R⁵)—R⁶ wherein
(a) R⁵ is hydrogen, alkyl-, cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 1-pyrrolidinylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶;
(b) R⁶ is
(1) cyano or Rs, wherein Rs is a [C₆ or C₁₀]aryl or a heterocycle containing 4-10 ring atoms of which 1-3 are heteroatoms selected from the group consisting of oxygen, nitrogen and sulfur;
(2) a group of the formula —W—Rs, wherein W is —C(═O)— or —S(O)ₙ— where n=1 or 2;
(3) a group of the formula —W—N(R⁹)R¹⁰, wherein
[a] R⁹ is hydrogen and R¹⁰ is an alkyl or cycloalkyl, optionally substituted by
(i) [C₆ or C₁₀]aryl, or
(ii) a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, thiomorpholin-4-yl, piperidin-1-yl, halo or (C₁-C₃)alkylenedioxy groups, or fused to a substituted phenyl or pyridine ring, wherein the ring fusion is at a carbon-carbon double bond of the heteroaryl ring, or
(iii) a heterocycle containing 4-10 ring atoms of which 1-3 are heteroatoms selected from the group consisting of oxygen, nitrogen and sulfur; or
[b] R⁹ is hydrogen or lower alkyl and R¹⁰ is Ar²; or
[c] R⁹ is hydrogen or lower alkyl, and R¹⁰ is a heterocycle containing 4-10 ring atoms of which 1-3 are heteroatoms are selected from the group consisting of oxygen, nitrogen and sulfur, said heterocycle; or
[d] R⁹ and R¹⁰ are both alkyl groups; or
[e] R⁹ and R¹⁰ together with N form a heterocycle containing 4-10 ring atoms which can incorporate up to one additional heteroatom selected from the group of N, O or S in the ring, wherein the heterocycle is optionally substituted with (C₆- or C₁₀)aryl, (C₆- or C₁₀)arylalkyl, or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy; or
[f] R⁹ and R¹⁰ are both hydrogen; and
c. X is a pharmaceutically acceptable anion, or
(B) a pharmaceutically acceptable salt of the compound,
wherein aryl or Ar* can be substituted with, in addition to any substitutions specifically noted, one or more general substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar²C(O)—, Ar²C(O)NH—, Ar²O—, Ar²—, Ar²-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid, 1-pyrrolidinyl, 4-[C₆ or C₁₀]arylpiperazin-1-yl-, 4-[C₆ or C₁₀]arylpiperidin-1-yl, azetidin-1-yl, morpholin-4-yl, thiomorpholin-4-yl, piperidin-1-yl;
wherein heterocycles, except those of Ar², can be substituted with, in addition to any substitutions specifically noted, the following general substitutions: acylamino, alkanoyl, alkoxy, alkoxycarbonyl, alkoxycarbonylalkyl, alkyl, alkylamino, alkylsulfonyl, alkylsulfinyl, alkylthio, amino, Ar²C(O)—, Ar²O—, Ar²—, carboxy, dialkylamino, fluoro, fluoroalkyl, difluoroalkyl, hydroxy, mercapto, sulfamoyl, or trifluoromethyl;
wherein the compound of formula VI differs from a salt of 3-[2-(4-bromophenyl)-2-oxoethyl]-1,3,4-thiadiazolium by one or more of the lack or replacement of the 4-bromo substitution, or the presence of one or more additional substitutions; and wherein the compound of formula VI differs from a salt of 3-(phenylmethyl)-1,3,4-thiadiazolium by the presence of one or more additional substitutions.

21. The compound of claim 20, wherein Y* is according to formula —CH(R⁵)—W—Rs.

23. The compound of claim 20, wherein
a. R¹¹ and R¹² are independently selected from hydrogen, acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, allyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid, alkylsulfonyl, alkylsulfinyl, alkylthio, trifluoromethyl, Ar², Ar²-alkyl, Ar²—O, Ar²SO₂—, Ar²SO—, Ar²S—, ArSO₂NH—, ARNH, (N—Ar²)(N-alkyl)N—, Ar²C(O)—, Ar²C(O)NH—, Ar²NH—C(O)—, and (N—Ar²)(N-alkyl)N—C(O)—; or
b. Y is a group of the formula —CH(R⁵)—R⁶ wherein
(a) R⁵ is hydrogen or alkyl;
(b) R⁶ is
(1) cyano or Rs;
(2) a group of the formula —W—Rs, wherein W is —C(═O)— or —S(O)ₙ— where n=1 or 2;
(3) a group of the formula —W—N(R⁹)R¹⁰, wherein
[a] R⁹ is hydrogen and R¹⁰ is an alkyl or cycloalkyl, optionally substituted by
(i) [C₆ or C₁₀]aryl, or
(ii) a 5- or 6-membered heteroaryl ring, wherein the optional substitutions on the heteroaryl ring are, in addition to the general substitutions, one or more halo or (C₁-C₃)alkylenedioxy groups, or form a fused a substituted phenyl, or
(iii) a heterocycle containing 4-10 ring atoms; or
[b] R⁹ is hydrogen or lower alkyl and R¹⁰ is Ar²; or
[c] R⁹ is hydrogen or lower alkyl, and R¹⁰ is a heterocycle containing 4-10 ring atoms; or
[d] R⁹ and R¹⁰ are both alkyl groups; or
[e] R⁹ and R¹⁰ together with N form a heterocycle containing 4-10 ring atoms, wherein each heteroaryl thereon can, in addition to the general substitutions, be optionally substituted with one or more halo or (C₁-C₃)alkylenedioxy; or
[f] R⁹ and R¹⁰ are both hydrogen; and
g. X is a pharmaceutically acceptable anion, or
(B) a pharmaceutically acceptable salt of the compound,
wherein aryl or Ar² can be substituted with, in addition to any substitutions specifically noted, one or more general substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar²C(O)—, Ar²C(O)NH—, Ar²O—, Ar²—, Ar-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid; and
wherein heterocycles, except those of Ar², can be substituted with, in addition to any substitutions specifically noted, the following general substitutions: acylamino, alkanoyl, alkoxy, alkoxycarbonyl, alkoxycarbonylalkyl, alkyl, alkylsulfonyl, alkylsulfmyl, alkylthio, Ar²C(O)—, Ar²O—, Ar²—, carboxy, fluoro, fluoroalkyl, difluoroalkyl, hydroxy, mercapto, sulfamoyl, or trifluoromethyl.

24. The compound of claim 23, wherein Y* is according to formula —CH(R⁵)—W—Rs.

25. A compound of claim 20 selected from:
5-Amino-3-carbamoylmethyl-[1,3,4]-thiadiazolium bromide;
2-Amino-3-(4-chloro-benzyl)-[1,3,4]-thiadiazolium chloride; and
2-Amino-3-(4-fluro-benzyl)-[1,3,4]-thiadiazolium bromide.

26. A pharmaceutical composition comprising:
a compound of one of claims 20 to 25; and
a pharmaceutically acceptable excipient.

27. A method of treating an indication of the invention with a pharmaceutically effective amount of a compound of one of claims 20 to 25.

28. A compound of formula VI: 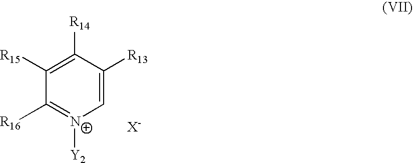
wherein
a. R¹³, R¹⁴, R¹⁵ and R¹⁶
1. are independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar³ (wherein Ar³ is C₆ or C₁₀ aryl), Ar³-alkyl, Ar³—O, Ar³SO₂—, Ar³SO—, Ar³S—, Ar³SO₂NH—, Ar³NH, (N—Ar³)(N-alkyl)N—, Ar³C(O)—, Ar³C(O)NH—, Ar³NH—C(O)—, and (N—Ar³)(N-alkyl)N—C(O)—, or together R₁ and R₂ comprise methylenedioxy; or
2. form, with an adjacent pair from R¹³, R¹⁴, R¹⁵ and R¹⁶, together with their ring carbons, a C₆- or C₁₀-aromatic fused ring system; or
3. form, with an adjacent pair from R¹³, R¹⁴, R¹⁵ and R¹⁶, together with their ring carbons, a C₅-C₇ fused cycloalkyl ring having up to two double bonds including the fused double bond of the pyridinium containing ring, which cycloalkyl ring can be substituted by one or more of the group consisting of alkyl, alkoxycarbonyl, amino, aminocarbonyl, carboxy, fluoro, or oxo substituents; or
4. form, with an adjacent pair from R¹³, R¹⁴, R¹⁵ and R¹⁶, together with their ring carbons, a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring may be optionally substituted with one or more 1-pyrrolidinyl-, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy groups; or
5. form, with an adjacent pair from R¹³, R¹⁴, R¹⁵ and R¹⁶ together with their ring carbons, a five to eight membered heterocycle, wherein the heterocycle consists of ring atoms selected from the group consisting of carbon, nitrogen, and S(O)ₙ, where n=0,1, or 2;
b. Y² is a group of the formula —CH(R⁵)—R⁶ wherein
(a) R⁵ is hydrogen, alkyl-, cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 1-pyrrolidin-1-ylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶;
(b) R⁶ is phenyl substituted at the para position with chloro or fluoro;
(2) a group of the formula —W—Rs, wherein W is —C(═O)— or —S(O)ₙ— where n=1 or 2;
(3) a group of the formula —W—N(R⁹)R¹⁰, wherein
[a] R⁹ is hydrogen and R¹⁰ is an alkyl or cycloalkyl, optionally substituted by
(i) [C₆ or C₁₀]aryl, or
(ii) a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, thiomorpholin-4-yl, piperidin-1-yl, halo or (C₁-C₃)alkylenedioxy groups, or fused to a phenyl or pyridine ring, wherein the ring fusion is at a carbon-carbon double bond of the heteroaryl ring, or
(iii) a heterocycle containing 4-10 ring atoms of which 1-3 are heteroatoms selected from the group consisting of oxygen, nitrogen and sulfur; or
[b] R⁹ is hydrogen or lower alkyl and R¹⁰ is Ar³; or
[c] R⁹ is hydrogen or lower alkyl, and R¹⁰ is a heterocycle containing 4-10 ring atoms of which 1-3 are heteroatoms are selected from the group consisting of oxygen, nitrogen and sulfur, said heterocycle; or
[d] R⁹ and R¹⁰ are both alkyl groups; or
[e] R⁹ and R¹⁰ together with N form a heterocycle containing 4-10 ring atoms which can incorporate up to one additional heteroatom selected from the group of N, O or S in the ring, wherein the heterocycle is optionally substituted with (C₆- or C₁₀)aryl, (C₆- or C₁₀)arylalkyl, or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy; or
[f] R⁹ and R¹⁰ are both hydrogen;
c. X is a pharmaceutically acceptable anion, or
(B) a pharmaceutically acceptable salt of the compound,
wherein aryl or Ar³ can be substituted with, in addition to any substitutions specifically noted, one or more general substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar³C(O)—, Ar³C(O)NH—, Ar³O—, Ar³-, Ar³-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid, 1-pyrrolidinyl, 4-[C₆ or C₁₀]arylpiperazin-1-yl-, 4-[C₆ or C₁₀]arylpiperidin-1-yl, azetidin-1-yl, morpholin-4-yl, thiomorpholin-4-yl, piperidin-1-yl;
wherein heterocycles, except those of Ar³, can be substituted with, in addition to any substitutions specifically noted, the following general substitutions: acylamino, alkanoyl, alkoxy, alkoxycarbonyl, alkoxycarbonylalkyl, alkyl, alkylamino, alkylsulfonyl, alkylsulfinyl, alkylthio, amino, Ar³C(O)—, Ar³O—, Ar³—, carboxy, dialkylamino, fluoro, fluoroalkyl, difluoroalkyl, hydroxy, mercapto, sulfamoyl, or trifluoromethyl;
wherein, if the compound of formula VII has a core structure comprising a pyridinium ring having a 2-aryl-2-oxoethyl substitution at the 1 position, wherein the aryl can be substituted, and a formyl which may be substituted at the 3 position, one or both of the following applies:
the compound of formula VII differs from a salt of pyridinium compound having a 1-(2-aryl-2-oxoethyl), wherein the aryl can be substituted, and a formyl which may be substituted at the 3 position by at least one additional substitution at R¹⁴, R¹⁵ or R¹⁶, or
the aryl of 2-aryl-2-oxoethyl is phenyl and is substituted at the para position with an electron withdrawing group selected from fluoro, chloro, nitro, trifluoromethyl, and carbamoyl; and
wherein the compound of formula VII differs from a salt of 1-[2-(4-methylphenyl)-2-oxoethyl]-pyridinium by one or more of the lack or replacement of the methyl substitution, or the presence of one or more additional substitutions.

29. The compound of claim 28, wherein Y² is according to formula —CH(R⁵)—W—Rs.

30. The compound of claim 28, wherein an adjacent pair from R¹³, R¹⁴, R¹⁵ and R¹⁶ together with their ring carbons, form a C₆- or C₁₀- aromatic fused ring which can be substituted by one or more halo, amino, alkyl, sulfonic acid, alkylsulfonyl or ω-alkylenesulfonic acid groups, or a C₁-C₃ alkylenedioxy group.

31. The compound of claim 28, wherein
a. R¹³, R¹⁴, R¹⁵ and R¹⁶
1. are independently selected from hydrogen, acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, allyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid, alkylsulfonyl, alkylsulfinyl, alkylthio, trifluoromethyl, Ar³, Ar³-alkyl, Ar³—O, Ar³SO₂—, Ar³SO—, Ar³S—, Ar³SO₂NH—, Ar³NH—, N—Ar³)(N-alkyl)—, Ar³C(O)—, Ar³C(O)NH—, ArNH—C(O)—, and (N—Ar)(N-alkyl)N—C(O)—; or
2. form, with an adjacent pair from R¹³, R¹⁴, R¹⁵ and R¹⁶, together with their ring carbons, a C₆- or C₁₀- aromatic fused ring system; or
3. form, with an adjacent pair from R¹³, R¹⁴, R¹⁵ and R¹⁶, together with their ring carbons, a C₅-C₇ fused cycloalkyl, which cycloalkyl ring can be substituted by one or more of the group consisting of alkyl, alkoxycarbonyl, aminocarbonyl, carboxy, fluoro, or oxo substituents; or
4. form, with an adjacent pair from R¹³, R¹⁴, R¹⁵ and R¹⁶ together with their ring carbons, a 5- or 6-membered heteroaryl ring, wherein each heteroaryl ring may, in addition to the general substitutions, be optionally substituted with one or more halo or (C₁-C₃)alkylenedioxy groups; or
5. form, with an adjacent pair from R¹³, R¹⁴, R¹⁵ and R¹⁶ together with their ring carbons, a five to eight membered heterocycle;
b. Y is a group of the formula —CH(R⁵)—-R⁶ wherein
(a) R⁵ is hydrogen or alkyl;
(b) R⁶ is
(1) cyano or Rs;
(2) a group of the formula —W—Rs, wherein W is —C(═O)— or —S(O)ₙ— where n=1 or 2;
(3) a group of the formula —W—N(R⁹)R¹⁰, wherein
[a] R⁹ is hydrogen and R¹⁰ is an alkyl or cycloalkyl, optionally substituted by
(i) [C₆ or C₁₀]aryl, or
(ii) a 5- or 6-membered heteroaryl ring, wherein said heteroaryl ring can, in addition to the general substitutions, be optionally substituted with one or more halo or (C₁-C₃)alkylenedioxy groups, or fused to a substituted phenyl, or
(iii) a heterocycle containing 4-10 ring atoms; or
[b] R⁹ is hydrogen or lower alkyl and R¹⁰ is Ar³; or
[c] R⁹ is hydrogen or lower alkyl, and R¹⁰ is a heterocycle; or
[d] R⁹ and R¹⁰ are both alkyl groups; or
[e] R⁹ and R¹⁰ together with N form a heterocycle, wherein each heteroaryl thereon can, in addition to the general substitutions, be optionally substituted with one or more halo or (C₁-C₃)alkylenedioxy; or
[f] R⁹ and R¹⁰ are both hydrogen; and
g. X is a pharmaceutically acceptable anion, or
(B) a pharmaceutically acceptable salt of the compound,
wherein aryl or Ar³ can be substituted with, in addition to any substitutions specifically noted, one or more general substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar³C(O)—, Ar³C(O)NH—, Ar³O—, Ar³—, Ar³-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid; and
wherein heterocycles, except those of Ar, can be substituted with, in addition to any substitutions specifically noted, the following general substitutions: acylamino, alkanoyl, alkoxy, alkoxycarbonyl, alkoxycarbonylalkyl, alkyl, alkylsulfonyl, alkylsulfinyl, alkylthio, Ar³C(O)—, Ar³O—, Ar³—, carboxy, fluoro, fluoroalkyl, difluoroalkyl, hydroxy, mercapto, sulfamoyl, or trifluoromethyl.

32. The compound of claim 31, wherein Y² is according to formula —CH(R⁵)—W—Rs.

33. A compound of claim 28, selected from:
3-(aminocarbonyl)-1-[2-(4-chlorophenyl)-2-oxoethyl]pyridinium chloride;
3-(aminocarbonyl)-1-benzylpyridinium bromide;
3-Carbamoyl-1-(4-methoxy-benzyl)-pyridinium chloride; and
3-Carbamoyl-1-[2-(4-fluoro-phenyl)-2-oxo-ethyl]-pyridinium chloride.

34. A pharmaceutical composition comprising:
a compound of one of claims 28 to 33; and
a pharmaceutically acceptable excipient.

35. A method of treating an indication of the invention with a pharmaceutically effective amount of a compound of one of claims 28 to 33.

36. A compound of formula VIII: 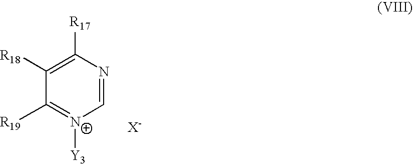
wherein
a. R¹⁷, R¹⁸ and R¹⁹
1. are independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁴ (wherein Ar² is C₆ or C₁₀ aryl), Ar⁴-alkyl, Ar⁴—O, Ar⁴SO₂—, Ar⁴SO—, Ar⁴S—, Ar⁴SO₂NH—, Ar⁴NH, (N—Ar⁴)(N-alkyl)N—, Ar⁴C(O)—, Ar⁴C(O)NH—, Ar⁴NH—C(O)—, and (N—Ar⁴)(N-alkyl)N—C(O)—, or together R¹ and R₂ comprise methylenedioxy; or
2. form, with an adjacent pair from R¹⁷, R¹⁸ and R¹⁹, together with their ring carbons, a C₆- or C₁₀- aromatic fused ring system; or
3. form, with an adjacent pair from R¹⁷, R¹⁸ and R¹⁹, together with their ring carbons, a C₅-C₇ fused cycloalkyl ring having up to two double bonds including the fused double bond of the pyridinium containing ring, which cycloalkyl ring can be substituted by one or more of the group consisting of alkyl, alkoxycarbonyl, amino, aminocarbonyl, carboxy, fluoro, or oxo substituents; or
4. form, with an adjacent pair from R¹⁷, R¹⁸ and R¹⁹, together with their ring carbons, a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring may be optionally substituted with one or more 1-pyrrolidinyl-, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy groups; or
5. form, with an adjacent pair from R¹⁷, R¹⁸ and R¹⁹, together with their ring carbons, a five to eight membered heterocycle, wherein the heterocycle consists of ring atoms selected from the group consisting of carbon, nitrogen, and S(O)ₙ, where n=0,1, or 2;
b. Y³ is a group of the formula —CH(R⁵)-R⁶ wherein
(a) R⁵ is hydrogen, alkyl-, cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 1-pyrrolidin-1-ylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶;
(b) R⁶ is phenyl substituted on the para position with chloro or fluoro;
c. X is a pharmaceutically acceptable anion, or
(B) a pharmaceutically acceptable salt of the compound,
wherein aryl (including phenyl) or Ar⁴ can be substituted with, in addition to any substitutions specifically noted, one or more general substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar⁴C(O)—, Ar⁴C(O)NH—, Ar⁴O—, Ar⁴—, Ar⁴-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid, 1-pyrrolidinyl, 4-[C₆ or C₁₀]arylpiperazin-1-yl-, 4-[C₆ or C₁₀]arylpiperidin-1-yl, azetidin-1-yl, morpholin-4-yl, thiomorpholin-4-yl, piperidin-1-yl; and
wherein heterocycles, except those of Ar⁴, can be substituted with, in addition to any substitutions specifically noted, the following general substitutions: acylamino, alkanoyl, alkoxy, alkoxycarbonyl, alkoxycarbonylalkyl, alkyl, alkylamino, alkylsulfonyl, alkylsulfinyl, alkylthio, amino, Ar⁴C(O)—, Ar⁴O—, Ar⁴—, carboxy, dialkylamino, fluoro, fluoroalkyl, difluoroalkyl, hydroxy, mercapto, sulfamoyl, or trifluoromethyl.

37. The compound of claim 36, wherein Y² is according to formula —CH(R⁵)—W—Rs.

38. The compound of claim 36, wherein an adjacent pair from R¹⁷, R¹⁸ and R¹⁹, together with their ring carbons, form a C₆- or C₁₀- aromatic fused ring which can be substituted by one or more halo, amino, alkyl, sulfonic acid, alkylsulfonyl or ω-alkylenesulfonic acid groups, or a C₁-C₃ alkylenedioxy group.

39. The compound of claim 36, wherein
a. R¹⁷, R¹⁸ and R¹⁹
1. are independently selected from hydrogen, acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, allyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid, alkylsulfonyl, alkylsulfinyl, alkylthio, trifluoromethyl, Ar⁴, Ar⁴-alkyl, Ar⁴—O, Ar⁴SO₂—, Ar⁴SO—, Ar⁴S—, Ar⁴SO₂NH—, Ar⁴NH, (N—Ar⁴)(N-alkyl)N—, Ar⁴C(O)—, Ar⁴C(O)NH—, Ar⁴NH—C(O)—, and (N—Ar⁴)(N-alkyl)N—C(O)—; or
2. form, with an adjacent pair from R¹⁷, R¹⁸ and R¹⁹, together with their ring carbons, a C₆- or C₁₀- aromatic fused ring system; or
3. form, with an adjacent pair from R¹⁷, R¹⁸ and R¹⁹, together with their ring carbons, a C₅-C₇ fused cycloalkyl ring; or
4. form, with an adjacent pair from R¹⁷, R¹⁸ and R¹⁹, together with their ring carbons, a 5- or 6-membered heteroaryl ring, wherein each heteroaryl ring may, in addition to the general substitutions, be optionally substituted with one or more halo or (C₁-C₃)alkylenedioxy groups; or
5. form, with an adjacent pair from R¹⁷, R¹⁸ and R¹⁹, together with their ring carbons, a five to eight membered heterocycle; and
c. X is a pharmaceutically acceptable anion, or
(B) a pharmaceutically acceptable salt of the compound,
wherein aryl or Ar⁴ can be substituted with, in addition to any substitutions specifically noted, one or more substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar⁴C(O)—, Ar⁴C(O)NH—, Ar⁴O—, Ar⁴—, Ar⁴-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid;
wherein heterocycles, except those of Ar⁴, can be substituted with, in addition to any substitutions specifically noted, acylamino, alkanoyl, alkoxy, alkoxycarbonyl, alkoxycarbonylalkyl, alkyl, alkylsulfonyl, alkylsulfinyl, alkylthio, Ar⁴C(O)—, Ar⁴O—, Ar⁴—, carboxy, fluoro, fluoroalkyl, difluoroalkyl, hydroxy, mercapto, sulfamoyl, or trifluoromethyl.

40. The compound of claim 39, wherein if Y has a core structure of phenyl substituted at the para position with chloro, then the compound of formula VIII differs from a salt of 1-[2-(4-bromophenyl)-2-oxoethyl]-5-cyano-pyrimidinium by a substitution difference of more than the cyano (which is not within the scope of R¹⁸).

41. The compound of claim 39, wherein Y² is according to formula —CH(R⁵)—W—Rs.

42. A compound of claim 36 selected from:
1-(4-Fluoro-benzyl)-pyrimidin-1-ium bromide; and
1-(4-chloro-benzyl)-pyrimidin-1-ium chloride.

43. A pharmaceutical composition comprising:
a compound of one of claims 36 to 42; and
a pharmaceutically acceptable excipient.

44. A method of treating an indication of the invention with a pharmaceutically effective amount of a compound of one of claims 36 to 42.

45. A compound of formula IX: 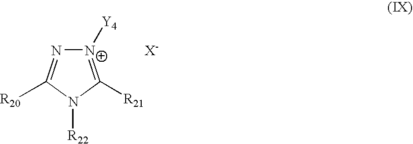
wherein
a. one of R²⁰ and R²¹ is hydrogen, and the other is selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁵ (wherein Ar² is C₆ or C₁₀ aryl), Ar⁵-alkyl, Ar⁵—O, Ar⁵SO₂—, Ar⁵SO—, Ar⁵S—, Ar⁵SO₂NH—, Ar⁵NH, (N—Ar⁵)(N-alkyl)N—, Ar⁵C(O)—, Ar⁵C(O)NH—, Ar⁵NH—C(O)—, or (N—Ar⁵)(N-alkyl)N—C(O)—;
b. R²² is acylamino, acyloxyalkyl, alkanoylalkyl, alkenyl, alkoxycarbonyl, alkoxycarbonylalkyl, alkyl, allyl, carbamoyl, carboxyalkyl, dialkylamino, (C₂-C₆)hydroxyalkyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁵, Ar⁵-alkyl, Ar⁵—O, Ar⁵SO₂—, Ar⁵SO—, Ar⁵S—, Ar⁵SO₂NH—, Ar⁵NH, (N—Ar⁵)(N-alkyl)N—, Ar⁵C(O)—, Ar⁵C(O)NH—, Ar⁵NH—C(O)—, or (N—Ar⁵)(N-alkyl)N—C(O)—;
c. Y⁴ is a group of the formula —CH(R⁵)-R⁶ wherein
(a) R⁵ is hydrogen, alkyl-, cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 1-pyrrolidinylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶;
(b) R⁶ is
(1) cyano;
(2) a group of the formula —W—Rs, wherein W is —C(═O)— or —S(O)ₙ— where n=1 or 2, and wherein Rs is a [C₆ or C₁₀]aryl or a heterocycle containing 4-10 ring atoms of which 1-3 are heteroatoms selected from the group consisting of oxygen, nitrogen and sulfur; and
d. X is a pharmaceutically acceptable anion, or
(B) a pharmaceutically acceptable salt of the compound,
wherein aryl or Ar⁵ can be substituted with, in addition to any substitutions specifically noted, one or more general substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar⁵C(O)—, Ar⁵C(O)NH—, Ar¹O—, Ar⁵—, Ar⁵-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid, 1-pyrrolidinyl, 4-[C₆ or C₁₀]arylpiperazin-1-yl-, 4-[C₆ or C₁₀]arylpiperidin-1-yl, azetidin-1-yl, morpholin-4-yl, thiomorpholin-4-yl, piperidin-1-yl; and
wherein heterocycles, except those of Ar⁵, can be substituted with, in addition to any substitutions specifically noted, the following general substitutions: acylamino, alkanoyl, alkoxy, alkoxycarbonyl, alkoxycarbonylalkyl, alkyl, alkylamino, alkylsulfonyl, alkylsulfinyl, alkylthio, amino, Ar⁵C(O)—, Ar⁵O—, Ar⁵—, carboxy, dialkylamino, fluoro, fluoroalkyl, difluoroalkyl, hydroxy, mercapto, sulfamoyl, or trifluoromethyl.

46. The compound of claim 45, wherein Y⁴ is according to formula —CH(R⁵)—W—Rs.

47. The compound of claim 45, wherein
a. R²⁰ and R²¹ are independently selected from hydrogen, acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, allyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid, alkylsulfonyl, alkylsulfinyl, alkylthio, trifluoromethyl, Ar², Ar²-alkyl, Ar²—O, Ar²SO₂—, Ar²SO—, Ar²S—, Ar²SO₂NH—, Ar²NH, (N—Ar²)(N-alyl)N—, Ar²C(O)—, Ar²C(O)NH—, Ar²NH—C(O)—, and (N—Ar²)(N-alkyl)N—C(O)—; or
b. Y is a group of the formula —CH(R⁵)—R⁶ wherein
(a) R⁵ is hydrogen or alkyl;
(b) R⁶ is
(1) cyano;
(2) a group of the formula —W—Rs, wherein W is —C(═O)— or —S(O)ₙ— where n=1 or 2; and
g. X is a pharmaceutically acceptable anion, or
(B) a pharmaceutically acceptable salt of the compound,
wherein aryl or Ar² can be substituted with, in addition to any substitutions specifically noted, one or more general substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar²C(O)—, Ar²C(O)NH—, Ar²O—, Ar²-, Ar²-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid; and
wherein heterocycles, except those of Ar², can be substituted with, in addition to any substitutions specifically noted, the following general substitutions: acylamino, alkanoyl, alkoxy, alkoxycarbonyl, alkoxycarbonylalkyl, alkyl, alkylsulfonyl, alkylsulfinyl, alkylthio, Ar²C(O)—, Ar²O—, Ar²—, carboxy, fluoro, fluoroalkyl, difluoroalkyl, hydroxy, mercapto, sulfamoyl, or trifluoromethyl.

48. The compound of claim 47, wherein Y* is according to formula —CH(R⁵)—W—Rs.

49. A pharmaceutical composition comprising:
a compound of one of claims 45 to 48; and
a pharmaceutically acceptable excipient.

50. A method of treating an indication of the invention with a pharmaceutically effective amount of a compound of one of claims 45 to 48.
